(19) United States
(12) Patent Application Publication
(10) Pub. No.: US 2026/0115140 A1
(43) Pub. Date: Apr. 30, 2026

(54) COMPOSITIONS FOR ENHANCED MUCOSAL PENETRATION

(71) Applicant: SHIN NIPPON BIOMEDICAL LABORATORIES, LTD., Kagoshima (JP)

(72) Inventor: Shunji HARUTA, Kagoshima (JP)

(21) Appl. No.: 19/401,051

(22) Filed: Nov. 25, 2025

Related U.S. Application Data
(63) Continuation-in-part of application No. PCT/IB2024/000291, filed on May 30, 2024.
(60) Provisional application No. 63/505,344, filed on May 31, 2023.

(51) Int. Cl. A61K 9/16; A61K 9/00; A61K 31/137; A61K 31/197; A61K 31/198; A61K 31/7088; A61K 38/13; A61K 38/23

(52) U.S. Cl. CPC A61K 9/1617; A61K 9/0043; A61K 9/1611; A61K 9/1623; A61K 9/1652; A61K 31/137; A61K 31/197; A61K 31/198; A61K 31/7088; A61K 38/13; A61K 38/23

(57) ABSTRACT

Provided herein are compositions and methods related to a solid dosage form comprising an absorption enhancer. The composition enhances the mucosal penetration and absorption of active ingredients.

### CROSS REFERENCE TO RELATED APPLICATIONS

This application is a continuation-in-part application of International Application No. PCT/IB2024/000291, filed on May 30, 2024, which claims the benefit of U.S. Provisional Application No. 63/505,344, filed on May 31, 2023, the entirety of which are incorporated herein by reference.

### BACKGROUND

Drug delivery via the mucosal surfaces, including the airways, the gastrointestinal (GI) tract, and the genital tract, may provide a desirable route of administration for delivery of numerous medications. Mucosal surfaces are often highly vascularized and may allow for bypassing of and first-pass metabolism in the gastrointestinal tract and liver. Furthermore, mucosal administration may allow for faster onset of action than oral administration. Mucosal delivery represents an advantageous option for achieving rapid and efficient systemic drug absorption, particularly for medications where quick therapeutic action is crucial.

### SUMMARY

Mucosal administration provides an alternative to invasive and typically painful delivery by injection and may allow for improved delivery of a medication to the brain by helping to overcome difficulties associated with passing the blood-brain barrier. Despite recent progress in pharmaceutical sciences, developing formulations with enhanced mucosal penetration or absorption remains challenging. For example, many nasal formulations face challenges associated with solubility, mucoadhesion, and mucopenetration, to name a few. Accordingly, there is a need for the development of novel formulations for use in nasal administration.

In some aspects, the present disclosure provides a pharmaceutical composition comprising: a) an active ingredient or a pharmaceutically acceptable salt thereof; b) an absorption enhancer comprising a choline ester; and c) an excipient comprising microcrystalline cellulose; wherein the pharmaceutical composition is formulated in a solid dosage form. In some embodiments, the choline ester is represented by the formula [(CH3)3N+CH2CH2OR]X—, wherein X is a pharmaceutically acceptable counterion. In some embodiments, X comprises a chloride. In some embodiments, R is selected from the group consisting of a C2-C20 alkyl, a C2-C20 alkenyl, a C2-C20 alkynyl, and a C2-C20 heteroalkyl. In some embodiments, the choline ester comprises a C8 to C20 alkanoylcholine selected from the group consisting of: an arachidoyl choline (C20), a stearoyl choline (C18), a palmitoyl choline (C16), a myristoylcholine (C14), a lauroylcholine (C12), a caproyl choline (C10), and a capryloyl choline (C8), and any combination thereof. In some embodiments, the choline ester comprises a palmitoylcholine (C16), a myristoylcholine (C14), or a lauroylcholine (C12). In some embodiments, pharmaceutical composition further comprises an additional excipient. In some embodiments, the additional excipient comprises chitosan, lactose, croscarmellose sodium, tribasic calcium phosphate, or any combination thereof.

In some embodiments, the pharmaceutical composition has an improved mucosal absorption rate compared to a corresponding pharmaceutical composition that does not comprise the absorption enhancer, the improved mucosal absorption rate being measured by a pharmacokinetic (PK) parameter of the active ingredient or a metabolite thereof. In some embodiments, the PK parameter of the active ingredient or a metabolite thereof improves by at least about 15% compared to a corresponding composition that does not comprise the absorption enhancer. In some embodiments, the pharmacokinetic (PK) parameter of the active ingredient or a metabolite thereof improves by at least about: 20%, 50%, 80%, 100%, 150%, 200%, 250%, 300%, 350%, 400%, 450%, 500%, 550%, 600%, 650%, 700%, 750%, or 800%, compared to a corresponding composition that does not comprise an absorption enhancer. In some embodiments, the improved PK parameter comprises a higher maximum drug concentration (Cmax). In some embodiments, the improved PK parameter comprises a shorter time to reach Cmax (Tmax). In some embodiments, the pharmaceutical composition has a Tmax of less than 10 minutes or less than 20 minutes. In some embodiments, the improved PK parameter comprises a higher bioavailability (F). In some embodiments, the improved PK parameter comprises a larger area under curve (AUC). In some embodiments, the AUC comprises AUC0-10 min, AUC0-20 min, AUC0-30 min, AUC0-60 min, AUC0-90 min, AUC0-120 min, AUC0-240 min, AUC0-480 min, or any combination thereof. In some embodiments, the improved PK parameter comprises an AUC that increases by at least about 200%, about 300%, about 400%, about 500%, or about 600% compared to a corresponding pharmaceutical composition that does not comprise an absorption enhancer. In some embodiments, the PK parameter is measured from plasma or cerebral spinal fluid (CSF). In some embodiments, the PK parameter is measured by a method comprising high performance liquid chromatography (HPLC), liquid chromatograph-mass spectrometry (LC-MS), enzyme immunoassay (EIA), enzyme-linked immunosorbent assay (ELISA), or any combination thereof.

In some embodiments, the excipient is present in an amount of at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, or at least about 70 mg. In some embodiments, the pharmaceutical composition provided herein comprises from about 10 mg to about 60 mg of the excipient. In some embodiments, the pharmaceutical composition comprises at least about 40%, at least about 45%, at least about 50%, at least about 55%, at least about 60%, at least about 65%, at least about 70%, at least about 75%, at least about 80% of the excipient by weight. In some embodiments, the pharmaceutical composition comprises from about 40% to about 65% of the excipient by weight. In some embodiments, the pharmaceutical composition comprises from about 70% to about 95% of the excipient by weight. In some embodiments, the microcrystalline cellulose has a mean particle size of from about 10 μm to about 100 μm. In some embodiments, the microcrystalline cellulose is present in about 40% of the total weight of the pharmaceutical composition. In some embodiments, the microcrystalline cellulose is present in about 90% of the total weight of the pharmaceutical composition. In some embodiments, the pharmaceutical composition further comprises an additional excipient, wherein the additional excipient comprises croscarmellose sodium or tribasic calcium phosphate.

In some embodiments, the active ingredient comprises a peptide. In some embodiments, the peptide comprises a dipeptide, a tripeptide, an oligopeptide, a polypeptide, or a cyclic peptide. In some embodiments, the active ingredient comprises a small molecule drug. In some embodiments, the active ingredient comprises a nucleic acid molecule. In some embodiments, the nucleic acid molecule comprises an antisense oligonucleotide. In some embodiments, the active ingredient comprises a middle molecule drug. In some embodiments, the active ingredient comprises epinephrine, baclofen, levodopa, calcitonin, cyclosporine A, an antisense oligonucleotide, or any combination thereof. In some embodiments, the active ingredient comprises epinephrine or a pharmaceutically acceptable salt thereof.

In some embodiments, the active ingredient comprises baclofen or a pharmaceutically acceptable salt thereof. In some embodiments, the active ingredient comprises levodopa or a pharmaceutically acceptable salt thereof. In some embodiments, the active ingredient comprises calcitonin or a pharmaceutically acceptable salt thereof. In some embodiments, the active ingredient comprises cyclosporine A or a pharmaceutically acceptable salt thereof. In some embodiments, the active ingredient or a pharmaceutically acceptable salt thereof is amorphous. In some embodiments, the active ingredient or a pharmaceutically acceptable salt thereof is crystalline.

In some embodiments, the pharmaceutical composition comprises from about 0.05 mg to about 30 mg of the active ingredient. In some embodiments, the pharmaceutical composition comprises a weight ratio of the absorption enhancer to microcrystalline cellulose of from about 1:1 to about 1:100. In some embodiments, the pharmaceutical composition comprises a weight ratio of the absorption enhancer to microcrystalline cellulose of from about 1:3 to about 1:15. In some embodiments, the pharmaceutical composition comprises from about 0.2 mg to about 20 mg of the absorption enhancer. In some embodiments, the pharmaceutical composition comprises from about 1.5 mg to about 10 mg of the absorption enhancer. In some embodiments, the pharmaceutical composition comprises at least about 0.2 mg, at least about 0.5 mg, at least about 1 mg, at least about 1.25 mg, at least about 1.5 mg, at least about 2 mg, at least about 2.5 mg, at least about 3 mg, at least about 3.5 mg, at least about 4 mg, at least about 4.5 mg, or at least about 5 mg of the absorption enhancer.

In some embodiments, the absorption enhancer is present in an amount of from about 1% to about 20% of a total weight of the pharmaceutical composition. In some embodiments, the absorption enhancer is present in an amount of from about 6% to about 15% of a total weight of the pharmaceutical composition. In some embodiments, a unit dose of the pharmaceutical composition weighs from about 20 mg to about 250 mg. In some embodiments, a unit dose of the pharmaceutical composition weighs from about 20 mg to about 80 mg. In some embodiments, a unit dose of the pharmaceutical composition weighs about 60 mg. In some embodiments, a unit dose of the pharmaceutical composition weighs about 80 mg.

In some embodiments, the pharmaceutical composition comprises about 1-5 mg of epinephrine or a pharmaceutically acceptable salt thereof, about 2-6 mg of a choline ester, and about 51-55 mg of microcrystalline cellulose. In some embodiments, the choline ester is lauroylcholine chloride. In some embodiments, the pharmaceutical composition has a Cmax of epinephrine or a metabolite thereof at least about: 150%, 250%, 350%, 450%, 550%, or 600% higher compared to a corresponding pharmaceutical composition that does not comprise said choline ester. In some embodiments, the pharmaceutical composition has an area under curve (AUC) of the epinephrine or a metabolite thereof at least about: 200%, 300%, 400%, 500%, 600%, 700%, or 750%% higher compared to a corresponding pharmaceutical composition that does not comprise said choline ester. In some embodiments, the AUC comprises AUC_{0-20 min}, AUC_{0-30 min}, AUC_{0-60 min}, AUC_{0-90 min}, AUC_{0-120 min}, or any combination thereof.

In some embodiments, the pharmaceutical composition comprises about 3-7 mg of baclofen or a pharmaceutically acceptable salt thereof, about 2-6 mg of a choline ester, and about 49-53 mg of the microcrystalline cellulose. In some embodiments, the choline ester is lauroylcholine chloride. In some embodiments, the pharmaceutical composition has a Cmax of the baclofen or a metabolite thereof at least about: 200%, 300%, or 400% higher compared to a corresponding pharmaceutical composition that does not comprise a choline ester. In some embodiments, the pharmaceutical composition has an AUC of the baclofen or a metabolite thereof at least about: 200%, 300%, 400%, or 500% higher compared to a corresponding pharmaceutical composition that does not comprise a choline ester. In some embodiments, the AUC comprises AUC_{0-20 min}, AUC_{0-30 min}, or AUC_{0-240 min} or any combination thereof.

In some embodiments, the pharmaceutical composition comprises about 18-28 mg of levodopa or a pharmaceutically acceptable salt thereof, about 2-6 mg of a choline ester, and about 20-50 mg of microcrystalline cellulose. In some embodiments, the pharmaceutical composition comprises about 18-28 mg of levodopa or a pharmaceutically acceptable salt thereof, about 2-6 mg of a choline ester, about 20-50 mg of microcrystalline cellulose, and, optionally, about 1-3 mg of croscarmellose sodium. In some embodiments, the choline ester is lauroylcholine chloride. In some embodiments, the pharmaceutical composition has a Cmax of the levodopa or a metabolite thereof at least about: 300%, 400%, 500%, or 600% higher compared to a corresponding pharmaceutical composition that does not comprise the choline ester. In some embodiments, the pharmaceutical composition has an AUC of levodopa or a metabolite thereof at least about: 300%, 400%, 500%, or 600% higher compared to a corresponding pharmaceutical composition that does not comprise the choline ester. In some embodiments, the AUC comprises AUC_{0-20 min}, AUC_{0-30 min}, AUC_{0-60 min}, or any combination thereof.

In some embodiments, the pharmaceutical composition comprises about 0.1-0.2 mg of calcitonin or a pharmaceutically acceptable salt thereof, about 2-6 mg of a choline ester, and about 54-57 mg of microcrystalline cellulose. In some embodiments, the choline ester is lauroylcholine chloride. In some embodiments, the pharmaceutical composition has a Cmax of the calcitonin or a metabolite thereof at least about 250% higher compared to a corresponding pharmaceutical composition that does not comprise the choline ester. In some embodiments, the pharmaceutical composition has an AUC of the calcitonin or a metabolite thereof at least about: 100%, 150%, 200%, 250%, or 300% higher compared to a corresponding pharmaceutical composition that does not comprise the choline ester. In some embodiments, the AUC comprises AUC_{0-20 min}, AUC_{0-30 min}, AUC_{0-120 min}, or any combination thereof.

In some embodiments, the pharmaceutical composition comprises about 28-32 mg of cyclosporine A or a pharmaceutically acceptable salt thereof, about 2-6 mg of a choline ester, and about 24-28 mg of microcrystalline cellulose. In some embodiments, the choline ester is lauroylcholine chloride. In some embodiments, the pharmaceutical composition has an AUC of the cyclosporine A or a metabolite thereof at least about: 600%, 700%, 800%, or 900% higher compared to a corresponding pharmaceutical composition that does not comprise the choline ester. In some embodiments, the AUC comprises AUC_{0-20 min}, AUC_{0-30 min}, AUC_{0-480 min}, or any combination thereof.

In some embodiments, the pharmaceutical composition comprises about 20-25 mg of the antisense oligonucleotide, about 7-10 mg of the choline ester, about 32-38 mg of microcrystalline cellulose, and about 0.3-0.6 mg of tribasic calcium phosphate. In some embodiments, the pharmaceutical composition has a Cmax of the nucleic acid molecule at least about 150% higher compared to a corresponding pharmaceutical composition that does not comprise the choline ester. In some embodiments, the pharmaceutical composition has an AUC of the nucleic acid molecule at least about 200% higher compared to a corresponding composition that does not comprise the choline ester In some embodiments, the AUC comprises AUC_{0-20 min}, AUC_{0-30 min}, AUC_{0-60 min}, AUC_{0-480 min}, or any combination thereof.

In some embodiments, the pharmaceutical composition is formulated for mucosal administration. In some embodiments, the mucosal administration comprises buccal administration, sublingual administration, oral administration, ocular administration, intranasal administration, rectal administration, or intravaginal administration. In some embodiments, the pharmaceutical composition is formulated for intranasal administration. In some embodiments, the solid dosage form comprises a powder dosage form, a tablet, a patch, or a capsule. In some embodiments, the solid dosage form comprises the powder dosage form.

Another aspect of the present disclosure includes a pharmaceutical composition provided herein for use in preventing, diagnosing, or treating a central nervous system disease. In some embodiments, the pharmaceutical composition is for use in a pre-operative or pre-examination procedure related to a central nervous system disease. In some embodiments, the pharmaceutical composition is for use in preventing, diagnosing, or treating a systemic disease. In some embodiments, the pharmaceutical composition is for use in preventing, diagnosing, or treating an infection.

Another aspect of the present disclosure includes a method of treating a disorder in a subject in need thereof, said method comprising administering to the subject the pharmaceutical compositions provided herein. In some embodiments, the disorder comprises a central nervous system (CNS) disorder. In some embodiments, the CNS disorder comprises cerebral hemorrhage, cerebral infarction, infections of the central nervous system, brain tumor, Parkinson's disease, epilepsy, amyotrophic lateral sclerosis, Alzheimer's disease, Lewy body dementia, progressive supranuclear palsy, corticobasal degeneration, Pick's disease, frontotemporal dementia, multiple sclerosis, schizophrenia, depression, bipolar disorder, dysthymia, adjustment disorder, anxiety disorders, agitation, panic disorder, obsessive-compulsive disorder, autism spectrum disorder, attention deficit/hyperactivity disorder, sleep disorder, insomnia, traumatic brain injury, glioma, pain, migraine, or any combination thereof. In some embodiments, the disorder comprises a systemic disorder. In some embodiments, the systemic disorder comprises anaphylaxis, defervescence, pain, inflammation, rheumatism, anxiety, psychosis, depression, epilepsy, Parkinson's disease, cerebral circulatory metabolic disorder, muscle tone disorder, autonomic neuropathy, dizziness, migraine, hypertension, angina, arrhythmia, cardiovascular diseases, allergies, asthma, bronchoconstriction, respiratory diseases, peptic ulcer, gastrointestinal disorders, diarrhea, indigestion, gout, hyperuricemia, dyslipidemia, diabetes, hormonal disorders, pituitary hormone disorder, corticosteroid disorder, sex hormone disorder, uterine related diseases, osteoporosis, bone metabolism diseases, obesity, vitamin deficiency, malnutrition, poisoning, cancer, hyperimmunity, autoimmune disorders, otorhinolaryngology related diseases, mouth related diseases, urinary/genital diseases, hemorrhoids, skin diseases, hematopoiesis/blood coagulation related diseases, narcotic dependence, lifestyle related diseases, or any combination thereof. In some embodiments, the disorder comprises Parkinson's disease, pain, migraine, anaphylaxis, epilepsy, anxiety, agitation, obesity, glioma, or Alzheimer's disease. In some embodiments, the subject comprises a human or a non-human primate. In some embodiments, the pharmaceutical composition is administered to the subject intranasally.

Another aspect of the present disclosure includes a method of enhancing the rate of absorption of an active ingredient or a pharmaceutically acceptable salt thereof, comprising administering to a subject the pharmaceutical compositions provided herein. In some embodiments, the administering comprises intranasal administration.

Another aspect of the present disclosure provides a method of making the pharmaceutical composition provided herein, comprising granulating a mixture of the active ingredient and the absorption enhancer of the pharmaceutical composition.

Another aspect of the present disclosure provides a method of treating a disease or condition in a subject in need thereof, said method comprising administering a pharmaceutical composition to said subject, wherein said pharmaceutical composition comprises an active agent for treating Parkinson's disease, and an absorption enhancer comprising a choline ester. In some embodiments, the pharmaceutical composition further comprises microcrystalline cellulose. In some embodiments, the pharmaceutical composition is in a solid dosage form. In some embodiments, the solid dosage form is a powder. In some embodiments, the administering comprises intranasal administration. In some embodiments, the disease or conditions comprises Parkinson's disease. In some embodiments, the active agent for treating Parkinson's disease comprises levodopa. In some embodiments, said administering produces a Tₘₐₓ of less than 20 minutes. In some embodiments, the administering produces a Tₘₐₓ of less than 15 minutes. In some embodiments, the administering produces a Tₘₐₓ of less than 10 minutes. In some embodiments, the administering produces an AUC of greater than 12000 ng×min/mL. In some embodiments, the administering produces a Cₘₐₓ of at least 300 ng/mL. In some embodiments, the administering produces a Cₘₐₓ of at least 500 ng/mL.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1F depict plasma epinephrine concentration-time profiles after intranasal administration of multiple different epinephrine preparations. FIG. 1A shows plasma epinephrine concentrations after intranasal administration of formulations comprising epinephrine alone or epinephrine in combination with an absorption enhancer. FIG. 1B shows plasma epinephrine concentrations after intranasal administration of formulations comprising epinephrine alone or epinephrine in combination with an excipient. FIG. 1C shows plasma epinephrine concentrations after intranasal administration of formulations comprising epinephrine, an absorption enhancer, and an excipient, or comprising epinephrine and either an absorption enhancer or an excipient. FIG. 1D shows plasma epinephrine concentrations after administration of a liquid formulation comprising epinephrine and lauroylcholine chloride (LCC), a powder formulation comprising epinephrine and chitosan, and a powder formulation comprising epinephrine, LCC, and chitosan. FIG. 1E shows plasma epinephrine concentrations after administration of formulations comprising epinephrine and LCC or comprising epinephrine, LCC, and lactose. FIG. 1F shows plasma epinephrine concentrations after administration of formulations comprising: 1) epinephrine, a choline ester (e.g., LCC, PCC, or MyCC), and MCC; 2) epinephrine and MCC; or 3) epinephrine, butyrylcholine iodide (BCI), and MCC.

FIGS. 2A-2E depict plasma baclofen concentration-time profiles after intranasal administration of baclofen preparations. FIG. 2A shows plasma baclofen concentrations after intranasal administration of Comparative Examples 12-16. Comparative Example 12 is a liquid baclofen formulation without an absorption enhancer or MCC. Comparative Examples 13-16 are liquid or powder formulations comprising baclofen and an absorption enhancer. FIG. 2B shows plasma baclofen concentrations after intranasal administration of formulations comprising baclofen alone, baclofen and MCC, or baclofen and lactose. FIG. 2C shows plasma baclofen concentrations after administration of different baclofen formulations (e.g., baclofen formulations comprising both an absorption enhancer and an excipient (e.g., MCC), baclofen formulations comprising either an absorption enhancer or MCC). FIG. 2D shows plasma baclofen concentrations after intranasal administration of baclofen formulations comprising both an absorption enhancer (e.g., LCC) and an excipient (e.g., lactose), or comprising either LCC or lactose. FIG. 2E shows plasma baclofen concentrations after intranasal administration of formulations comprising baclofen and an absorption enhancer, baclofen and an excipient, or baclofen, an absorption enhancer, and an excipient.

FIG. 3 depicts plasma levodopa concentration-time profiles after intranasal administration of various levodopa formulations (e.g., levodopa compositions comprising an absorption enhancer, and an excipient, or an excipient without an absorption enhancer).

FIG. 4 depicts plasma calcitonin concentration-time profiles after intranasal administration of various calcitonin formulations (e.g., calcitonin compositions comprising an absorption enhancer, and an excipient, or an excipient without an absorption enhancer).

FIG. 5 depicts blood cyclosporine A concentration-time profiles after intranasal administration of various cyclosporine formulations (e.g., cyclosporine compositions comprising an absorption enhancer, and an excipient, or an excipient without an absorption enhancer).

FIG. 6 depicts plasma antisense oligonucleotide concentration-time profiles after intranasal administration of two antisense oligonucleotide formulations (e.g., antisense oligonucleotide compositions comprising an antisense oligonucleotide (ASO), an absorption enhancer, and an excipient, or an ASO and an excipient without an absorption enhancer).

### DETAILED DESCRIPTION

Drug delivery via mucosal surfaces, including the airways, the gastrointestinal (GI) tract, and the genital tract, represents an alternative to invasive delivery by injection. Mucosal administration may offer several advantages, such as lower manufacturing and administration costs, potentially higher patient compliance—especially with subjects who suffer from a fear of needles—and a decreased risk of infection. Additionally, mucosal drug delivery via the nose, lungs, oral cavity, and genital tract can help circumvent hepatic first-pass metabolism. Mucosal surfaces are typically highly vascularized and easily accessible; however, drug delivery may be hindered by mucus, a complex viscoelastic gel that covers much of the soft tissue in the human body. The mucus layer, which contains large amounts of mucins (polymeric proteins densely decorated with negatively charged glycosaminoglycans) forms a highly entangled network with glycoproteins, lipids, and DNA. This network can prevent pathogens from invading but may also inhibit therapeutic agents from being quickly and adequately absorbed. This slow absorption may prolong the period during which subjects may benefit from a medication administered through a mucosal surface. Accordingly, there is a need for mucosal compositions that help overcome problems associated with absorption hindrances.

Provided herein, in some embodiments, are pharmaceutical compositions and methods related to a solid dosage form with improved mucosal penetration and/or absorption of active ingredients. In some embodiments, the compositions comprise an active ingredient or a pharmaceutically acceptable salt thereof and an absorption enhancer, wherein the absorption enhancer comprise a choline ester derived from a carboxylic acid. In some embodiments, the compositions comprise an active ingredient or a pharmaceutically acceptable salt thereof, an absorption enhancer, and an excipient comprising a microcrystalline cellulose. In some embodiments, the solid dosage form is a powder dosage form. The absorption enhancer of the composition may comprise a choline ester derived from a carboxylic acid or a pharmaceutically acceptable salt thereof. In some embodiments, the choline ester comprises an alkanoylcholine of various chain length. The alkanoylcholine can comprise a palmitoylcholine (PC), a myristoylcholine (MyC), or a lauroylcholine (LC), or a pharmaceutically acceptable salt thereof. In some embodiments, the excipient comprises microcrystalline cellulose (MCC). In some embodiments, the absorption enhancer is not derived from phosphatidic acid. In some embodiments, the absorption enhancer does not contain a phosphate group. In some embodiments, the absorption enhancer is not didecylphosphatidylcholine, lysolauroylphosphatidylcholine, dioctanoylphosphatidylcholine, dilauroylphosphatidylglycerol, or phosphatidylcholine. In some embodiments, the absorption enhancer is not phosphatidylcholine.

### Absorption Enhancer

In some aspects, the pharmaceutical composition comprises an absorption enhancer. An absorption enhancer may be a compound that enhance absorptions of an active ingredient. The term absorption enhancer may refer to an agent whose function is to increase absorption by enhancing membrane permeation, rather than increasing solubility. As such absorption enhancers are sometimes also referred to as permeation enhancers.

The absorption enhancer may improve mucosal penetration and/or absorption of the active ingredient via mucosal membranes, such as those found in nasal, buccal, sublingual, rectal, oracular, pulmonary, or vaginal mucosal membranes. In some embodiments, the absorption enhancer improves penetration and/or absorption of the active ingredient through the nasal mucus membrane. In some embodiments, the absorption enhancer increases the mucosal absorption rate. In some embodiments, the absorption enhancer improves potency, onset of action, half-life, or an amount of active ingredients absorbed through mucus membranes.

In some embodiments, an absorption enhancer comprises groups of compounds, such as, fatty acids, choline esters, surfactants, chelating agents, sugars, or any combination thereof.

In some embodiments, the absorption enhancer comprises a choline ester or a pharmaceutically acceptable salt thereof. A choline ester or a pharmaceutically acceptable salt thereof may comprise an ester of choline having the formula [(CH₃)₃N⁺CH₂CH₂OR]X, wherein X is a pharmaceutically acceptable counterion such as chloride, sulfate, nitrate, perchlorate, bromide, iodide, phosphate, acetate, benzoate, tartrate, citrate, propionate, gluconate, lactate, meleate, fumarate, bezylate, camsylate, esylate, gluceptate, mesylate, napsylate and the like; and wherein R is an acyl group. In some embodiments, the acyl group is derived from a carboxylic acid. In some embodiments, the acyl group comprises a C₂-C₂₀ alkyl, a C₂-C₂₀ alkenyl, a C₂-C₂₀ alkynyl, a C₂-C₂₀ heteroalkyl, a C₃-C₂₀ cycloalkyl, a C₃-C₂₀ heterocycloalkyl, an aryl, a heteroaryl, or any combination thereof. In some embodiments, the acyl group is selected from the group consisting of a C₂-C₂₀ alkyl, a C₂-C₂₀ alkenyl, a C₂-C₂₀ alkynyl, a C₂-C₂₀ heteroalkyl, a C₃-C₂₀ cycloalkyl, a C₃-C₂₀ heterocycloalkyl, an aryl, a heteroaryl, and any combination thereof. In some embodiments, the choline ester comprises an acyl choline. In some embodiments, the absorption enhancer does not contain a phosphate group. In some embodiments, the absorption enhancer is not didecylphosphatidylcholine, lysolauroylphosphatidylcholine, dioctanoylphosphatidylcholine, dilauroylphosphatidylglycerol, or phosphatidylcholine. In some embodiments, the absorption enhancer is not derived from phosphatidic acid. In some embodiments, the absorption enhancer is not phosphatidylcholine.

In some embodiments, the choline ester is derived from a carboxylic acid. In some embodiments, the choline ester comprises an acyl group from a carboxylic acid, wherein the acyl group comprises a C₂-C₂₀ alkyl, a C₂-C₂₀ alkenyl, a C₂-C₂₀ alkynyl, a C₂-C₂₀ heteroalkyl, a C₃-C₂₀ cycloalkyl, a C₃-C₂₀ heterocycloalkyl, an aryl, a heteroaryl, or any combination thereof. In some embodiments, the choline ester comprises the group selected from the group consisting of a C₂-C₂₀ alkyl, a C₂-C₂₀ alkenyl, a C₂-C₂₀ alkynyl, a C₂-C₂₀ heteroalkyl, a C₃-C₂₀ cycloalkyl, a C3-C20 heterocycloalkyl, an aryl, a heteroaryl, and any combination thereof. In some embodiments, the choline ester comprises a C₂-C₂₀ alkyl selected from the group consisting of a hexanoyl, an octanoyl, a decanoyl, a lauroyl, a myristoyl, a palmitoyl, or a stearoyl. In some embodiments, the choline ester comprises a C₂-C₂₀ alkenyl comprising one or more double bonds comprising 2-hexenoyl, 9-decenoyl, 9-hexadecenoyl, palmitoyl, oleioyl, myristoleoyl, 9, 12-hexadecadienoyl, linoleoyl, α-linoleoyl, γ-linolenoyl, arachidyl, or eicosapentaenoyl.

In some embodiments, the choline ester comprises an unsubstituted or a substituted acyl group. In some embodiments, the acyl group is substituted by at least one hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, or haloalkoxy.

In some embodiments, the choline ester comprises an alkanoylcholine or a pharmaceutically acceptable salt thereof. In some embodiments, the alkanoylcholine comprises a C₈ to C₂₀ alkyl selected from the group consisting of: arachidoyl choline (C20), stearoyl choline (C18), palmitoyl choline (C16), myristoylcholine (C14), lauroylcholine (C12), caproyl choline (C10), capryloyl choline (C8), and a pharmaceutically acceptable salt thereof. In some embodiments, the alkanoylcholine comprises a C₁₀ to C₁₈ alkyl. In some embodiments, the alkanoylcholine comprises a C₁₂ to C₁₆ alkyl. In some embodiments, the preferred alkanoylcholine comprises a palmitoyl choline (C16), a myristoylcholine (C14), or a lauroylcholine (C12), or a pharmaceutically acceptable salt thereof. In some embodiments, the most preferred alkanoylcholine is lauroylcholine (C12), or a pharmaceutically acceptable salt. In some embodiments, the preferred pharmaceutically acceptable salt of the absorption enhancer is a chloride salt.

In some embodiments, the absorption enhancer (e.g., choline ester) comprises an alkenoylcholine or a pharmaceutically acceptable salt thereof. In some embodiments, the alkenoylcholine comprises a C₂-C₂₀ alkenyl comprising 2-hexenoyl, 9-decenoyl, 9-hexadecenoyl, palmitoyl, oleioyl, myristoleoyl, 9, 12-hexadecadienoyl, linoleoyl, α-linoleoyl, γ-linolenoyl, arachidyl, or eicosapentaenoyl.

In some embodiments, the pharmaceutical composition provided herein comprises one or more absorption enhancers or a pharmaceutically acceptable salt thereof. In some embodiments, the pharmaceutical composition provided herein comprises at least 1, 2, 3, 4, 5, or more absorption enhancers or a pharmaceutically acceptable salt thereof. In some embodiments, the pharmaceutical composition provided herein comprises at most 1, 2, 3, 4, or 5 absorption enhancers or a pharmaceutically acceptable salt thereof.

In some embodiments, the preferred pharmaceutical composition comprises palmitoyl choline (C16), myristoylcholine (C14), or lauroylcholine (C12), or a pharmaceutically acceptable salt thereof. In some embodiments, the preferred absorption enhancer comprises palmitoylcholine chloride, myristoylcholine chloride, or lauroylcholine chloride (LCC). In some embodiments, the absorption enhancer comprises LCC. In some embodiments, the absorption enhancer comprises palmitoylcholine chloride (PCC). In some embodiments, the absorption enhancer comprises myristoylcholine chloride (MyCC).

In some embodiments, the pharmaceutical composition comprises at least: about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 6 mg, about 7 mg, about 8 mg, about 9 mg, about 10 mg, about 15 mg, or about 20 mg of an absorption enhancer. In some embodiments, the pharmaceutical composition comprises at most: about 1 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 6 mg, about 7 mg, about 8 mg, about 9 mg, about 10 mg, about 15 mg, or about 20 mg of an absorption enhancer. In some embodiments, the pharmaceutical composition comprises about 5 mg of the absorption enhancer. In some embodiments, the pharmaceutical composition comprises about 10 mg of the absorption enhancer.

In some embodiments, the pharmaceutical composition comprises from about 0.1 mg to about 30 mg of the absorption enhancer. In some embodiments, the pharmaceutical composition comprises from about 1 mg to about 20 mg, from about 1 mg to about 15 mg, from about 1 mg to about 10 mg, from about 1 mg to about 5 mg, from about 1 mg to about 2.5 mg, from about 1.5 mg to about 20 mg, from about 1.5 mg to about 15 mg, from about 1.5 mg to about 10 mg, from about 1.5 mg to about 5 mg, from about 1.5 mg to about 2.5 mg, from about 2 mg to about 20 mg, from about 2 mg to about 15 mg, from about 2 mg to about 10 mg, from about 2 mg to about 5 mg, from about 2 mg to about 2.5 mg, from about 3 mg to about 20 mg, from about 3 mg to about 15 mg, from about 3 mg to about 10 mg, from about 3 mg to about 5 mg, from about 5 mg to about 20 mg, from about 5 mg to about 15 mg, or from about 5 mg to about 10 mg of an absorption enhancer.

In some embodiments, the absorption enhancer of the pharmaceutical composition is present in at least: about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or at least about 20% of the total weight (w/w %) of the pharmaceutical composition. In some embodiments, the enhancer of the pharmaceutical composition is present in at most: about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or at most about 20% of the total weight (w/w %) of the pharmaceutical composition.

In some embodiments, the absorption enhancer of the pharmaceutical composition is present in from about 1% to about 20%, from about 1% to about 18%, from about 1% to about 15%, from about 1% to about 12%, from about 1% to about 10%, from about 1% to about 8%, from about 1% to about 5%, from about 3% to about 20%, from about 3% to about 18%, from about 3% to about 15%, from about 3% to about 12%, from about 3% to about 10%, from about 3% to about 8%, from about 3% to about 5%, from about 6% to about 20%, from about 6% to about 18%, from about 6% to about 15%, from about 6% to about 12%, from about 6% to about 10%, from about 6% to about 8%, from about 8% to about 20%, from about 8% to about 18%, from about 8% to about 15%, from about 8% to about 12%, from about 8% to about 10%, from about 12% to about 20%, from about 12% to about 18%, from about 12% to about 15%, from about 14% to about 20%, from about 14% to about 18%, from about 14% to about 15%, from about 16% to about 20%, from about 16% to about 18%, or from about 18% to about 20% of the total weight (w/w %) of the pharmaceutical composition.

In some embodiments, the absorption enhancer comprises at least about 3% (e.g., 3.7%) of the total weight (w/w %) of the pharmaceutical composition. In some embodiments, the absorption enhancer comprises at least about 6% (e.g., 6.2%, 6.7%, etc.) of the total weight (w/w %) of the pharmaceutical composition. In some embodiments, the absorption enhancer comprises at least about 8% (e.g., 8%) of the total weight (w/w %) of the pharmaceutical composition. In some embodiments, the absorption enhancer comprises at least about 10% (e.g., 13.3%) of the total weight (w/w %) of the pharmaceutical composition.

In some embodiments, the pharmaceutical composition comprising a choline ester (e.g., LCC) derived from a carboxylic acid and an excipient (e.g., microcrystalline cellulose (MCC)) demonstrates enhanced pharmacokinetic parameters compared with a corresponding pharmaceutical composition comprising another class of absorption enhancer (e.g., surfactant or sugar).

### Active Ingredient

The pharmaceutical composition provided herein comprises one or more active ingredients or pharmaceutically acceptable salts thereof.

In some embodiments, the active ingredient of the pharmaceutical composition may be a peptide drug or a peptide-related drug. In some embodiments, the peptide drug comprises a dipeptide, a tripeptide, an oligopeptide, a polypeptide or a cyclic peptide. In some embodiments, the peptide-related drug comprises a peptide mimetic, a peptide analog, or a drug-peptide conjugate. In some embodiments, the peptide or peptide-related drug have a molecular weight of about 10,000 Dalton (Da) or less, about 20,000 Da or less, about 30,000 Da or less, about 40,000 Da or less, or about 50,000 Da or less.

In some instances, the peptide or peptide-related drug comprises insulin, human growth hormone, calcitonin, glucagon, parathyroid hormone, para-thyroid hormone (1-34), glucagon-like peptide-1 (GLP-1), interferon, interleukin, erythropoietin, luteinizing hormone-releasing hormone, somatostatin, vasopressin, oxytocin, enkephalin, adrenocorticotropic hormone, growth hormone-releasing hormone, granulocyte colony formation-stimulating factor, parathyroid hormone, thyroid-stimulating hormone-releasing hormone, angiotensin, prolactin, luteinizing hormone, gastric inhibitory polypeptide (GIP), C-peptide, cyclosporine, FK-506, octreotide, carperitide, pramlintide, lanreotide, eptifibatide, albiglutide, pasireotide, teriparatide, exenatide, liraglutide, emfuvirtide, ziconotide, ecallantide, mifamurtide, nesiritide, peg-linesatide, afamelanotide, linaclotide, lixisenatide, teduglutide, bentiromide, cureletide diethylamine, degarelix, ghrelin, atrial natriuretic peptide, a peptide analog thereof, or any combination thereof.

In some embodiments, the active ingredient comprises a non-peptide drug.

In some embodiments, the active ingredient comprises a small molecule drug. In some embodiments, the small molecule drug has a molecular weight of less than about 1000 grams/mole (g/mol), about 750 g/mol, or about 500 g/mol. In some embodiments, the small molecule drug comprises epinephrine, baclofen, or levodopa, or a pharmaceutically acceptable salt thereof, or any combination thereof.

In some embodiments, the active ingredient comprises a middle molecule drug having a molecular weight of from about 500 g/mol to about 50,000 g/mol. In some embodiments, a middle molecule drug comprises a polysaccharide, a protein, or a nucleic acid molecule. In some embodiments, the middle molecule drug comprises calcitonin, cyclosporine A, or an antisense oligonucleotide, or a pharmaceutically acceptable salt thereof, or any combination thereof.

In some embodiments, the active ingredient comprises a drug having a molecular weight of over 50,000 g/mol comprising an antibody or a fragment thereof.

In some embodiments, the active ingredient comprises a nucleic acid molecule. In some embodiments, the nucleic acid molecule may be of various sizes comprising oligonucleotides, polynucleotides, or plasmids. In some embodiments, the nucleic acid molecule may be chemically modified nucleic acids comprising a modified backbone, a modified sugar moiety or a combination thereof.

In some embodiments, the nucleic acid molecule comprises an antisense oligonucleotide (e.g., an antisense oligomer or ASO). As used herein, the terms “ASO” and “antisense oligomer” are used interchangeably and refer to an oligomer such as a polynucleotide, comprising nucleobases that hybridizes to a target nucleic acid (e.g., a pre-mRNA or an mRNA) sequence by Watson-Crick base pairing or wobble base pairing (G-U).

In some embodiments, the antisense oligonucleotide comprises a phosphorothioate linkage or a phosphorodiamidate linkage. In some embodiments, each internucleotide linkage of the antisense oligonucleotide is a phosphorothioate linkage. In some embodiments, the antisense oligonucleotide comprises a phosphorodiamidate morpholino, a locked nucleic acid, a peptide nucleic acid, a 2′-O-methyl moiety, a 2′-Fluoro moiety, a 2′-O-methoxyethyl moiety, or a 2′-NMA moiety. In some embodiments, the antisense oligonucleotide comprises at least one modified sugar moiety. In some embodiments, each nucleotide of the antisense oligonucleotide comprises a modified sugar moiety. In some embodiments, each nucleotide of the antisense oligonucleotide comprises a 2′-O-methoxyethyl moiety. In some embodiments, the antisense oligonucleotide comprises at least one modified nucleobase. In some embodiments, the antisense oligonucleotide comprises hypoxanthine, xanthine, 7-methylguanine, 5, 6-dihydrouracil, 5-methylcytosine, or 5-hydroxymethoylcytosine.

In some embodiments, the antisense oligonucleotide consists of from 8 to 50 nucleobases, 8 to 40 nucleobases, 8 to 30 nucleobases, 8 to 25 nucleobases, 8 to 20 nucleobases, 8 to 15 nucleobases, 10 to 50 nucleobases, 10 to 40 nucleobases, 10 to 30 nucleobases, 10 to 25 nucleobases, 10 to 20 nucleobases, 10 to 15 nucleobases, 12 to 50 nucleobases, 12 to 40 nucleobases, 12 to 30 nucleobases, 12 to 25 nucleobases, 12 to 20 nucleobases, 12 to 15 nucleobases, 15 to 50 nucleobases, 15 to 40 nucleobases, 15 to 30 nucleobases, 15 to 25 nucleobases, 15 to 20 nucleobases, 15 to 19 nucleobases, 15 to 18 nucleobases, 15 to 16 nucleobases, 16 to 20 nucleobases, 16 to 19 nucleobases, 16 to 18 nucleobases, 17 to 20 nucleobases, 17 to 19 nucleobases, or 18 to 20 nucleobases.

In some embodiments, the active ingredient comprises acyclovir, adrenaline, alirocumab, allergen extract powder, amantadine, amphetamine, ampicillin, adalimumab, apomorphine, aripiprazole, atomoxetine, baclofen, beclomethasone, berimumab, betahistine, blonanserin, brexpiprazole, bromvalerylurea, buprenorphine, butorphanol, cabergoline, calcitonin, canakinumab, cannabide, cannabinoid, carbamazepine, carboplatin, cefaclor, ceftazidime, chimeric antibody abciximab, chloral hydrate, chlorpheniramine, chlorpromazine, cisplatin, clonazepam, codeine, cyanocobalamin, cyclophosphamide, cyclosporine A, cyclosporine, dantrolene, delavirdine, denosumab, desmopressin, dexamethasone, dextromethorphan, diazepam, didanosine, digoxin, dihydroergotamine, diltiazem, dimethyl fumarate, donepezil, droxidopa, edaravone, enoxacin, epinephrine, erenumab, erythropoietin, ethosuximide, etizolam, etoposide, evolocumab, fentanyl, fesoterodine, finasteride, fluorodeoxyglucose, fluorothymidine, fluphenazine, follicle stimulating hormone, fomivirsen, fremanezumab, gabapentin, galantamine, galcanezumab, ganciclovir, ghrelin, glucagon, glucagon-like peptide-1 (GLP-1), golimumab, granisetron, granulocyte colony stimulating factor, growth hormone releasing peptide, growth hormone, guanfasin, haloperidol, humanized antibody bevacizumab, hydromorphone, ibuprofen, ifosfamide, imidafenacin, indinavir, indisetron, insulin, insulin-like growth factor-1 (IGF-1), interferon, interleukin, iopamidol, ipilimumab, keratinocyte growth factor (KGF), laxibacumab, L-dopa, leptin, leuprorelin, levetiracetam, levodopa, loperamide, lormetazepam, low molecular weight heparin, loxoprofen, luteinizing hormone, manganese, melatonin, memantine, menopausal gonadotropic hormone, methylphenidate, midazolam, mipomersen, mirabegron, morphine, mouse antibody burinatumomab, naloxone, naphazoline, natalizumab, nelfinavir, nerve growth factor (NGF), nesitumumab, nevirabine, nevirapine, nitrazepam, nivolumab, nusinersen, ofatumumab, olanzapine, ondansetron, oxybutynin, oxycodone, oxytocin, ozagrel, palonosetron, panipenem-betamipron, panitumumab, pentobarbital, phenobarbital, phenytoin, piperiden, piperidene, pembrolizumab, placental gonadotropic hormone, primidone, progesterone, prolactin, propiverine, parathyroid hormone (PTH), PTH-related peptide, quetiapine, ramucirumab, remimazolam, ribavirin, rilmazaphone, risperidone, ritonavir, rivastigmine, rizatriptan, saquinavir, secukinumab, selective thrombin inhibitors, sildenafil, silodosin, simeprevir, sodium valproate, solifenacin, spiperone, stem cell growth factor (SCGF), sumatriptan, tacrolimus, tadalafil, tamsulosin, technesium, telaprevir, temozolomide, thallium, tiapride, tissue plasminogen activators, tolterodine, topiramate, tramadol, tranilast, triazolam, trihexyphenidyl, tropisetron, valganciclovir, vancomycin, vardenafil, vasopressin, vidarabine, vincristine, zalcitabine, zidovudine, zolmitriptan, zolpidem, zonisamide, zopiclone, 5-FU, or a pharmaceutically acceptable salt thereof, or a combination thereof.

In some embodiments, the active ingredient comprises epinephrine, baclofen, levodopa, calcitonin, cyclosporine A, or an antisense oligonucleotide, or a pharmaceutically acceptable salt thereof, or any combination thereof.

In some embodiments, the active ingredient comprises an agent having biological activity on the central nervous system (CNS). In some embodiments, the active ingredient comprises an agent effective for preventing or treating CNS diseases, for an examination or diagnosis of CNS diseases, or for a pre-operative or pre-examination treatment of CNS diseases. In some embodiments, the agent having biological activity on the CNS comprises levodopa or a pharmaceutically acceptable salt thereof, or any combination thereof.

In some embodiments, the active agent is not glucagon. In some embodiments, the active agent is not glucagon when the absorption enhancer is phosphatidylcholine. In some embodiments, the absorption enhancer is not derived from phosphatidic acid. In some embodiments, the absorption enhancer does not contain a phosphate group. In some embodiments, the absorption enhancer is not didecylphosphatidylcholine, lysolauroylphosphatidylcholine, dioctanoylphosphatidylcholine, dilauroylphosphatidylglycerol, or phosphatidylcholine. In some embodiments, the absorption enhancer is not phosphatidylcholine.

### Baclofen

In some embodiments, the active ingredient comprises an active agent for reducing muscle spasticity. In some embodiments, the active ingredient comprises a skeletal muscle relaxant. In some embodiments, the skeletal muscle relaxant is administered to treat spasticity generating from the spinal cord (e.g., spinal cord injury, multiple sclerosis, etc.). In some embodiments, the skeletal muscle relaxant is administered to treat spasticity generating from the brain (e.g., the cerebrum (e.g., cerebral palsy, traumatic brain injury, etc.)). In some embodiments, the skeletal muscle relaxant is administered to treat multiple sclerosis. In some embodiments, the skeletal muscle relaxant is administered to treat a spinal cord injury or a spinal cord disease. In some embodiments, the skeletal muscle relaxant is administered to treat dystonia. In some embodiments, the skeletal muscle relaxant is administered to treat spasticity associated with cerebral palsy. In some embodiments, the skeletal muscle relaxant is administered to treat trigeminal neuralgia. In some embodiments, the skeletal muscle relaxant is administered to treat complex regional pain syndrome. In some embodiments, the skeletal muscle relaxant comprises baclofen.

In some embodiments, the active ingredient comprises a drug for treating pain. In some embodiments, the drug for treating pain comprises baclofen.

### Levodopa

In some embodiments, the active ingredient comprises an active agent for treating Parkinson's disease. In some embodiments, the active ingredient comprises an active agent for treating postencephalitic parkinsonism or symptomatic parkinsonism. In some embodiments, the active ingredient comprises an active agent for treating motor fluctuations (e.g., such as off-episodes in advanced Parkinson's disease). In some embodiments, the active ingredient comprises a precursor to a neurotransmitter. In some embodiments, the active ingredient comprises a dopamine precursor. In some embodiments, the active agent comprises a dopamine agonist. In some embodiments, the active ingredient comprises a levodopa.

### Epinephrine

In some embodiments, the active ingredient comprises an active agent for treating type I allergic reactions, such as anaphylaxis. In some embodiments, the active ingredient comprises an active agent for treating hypotension associated with septic shock. In some embodiments, the active ingredient comprises an active agent that increases the mean arterial blood pressure and/or induces bronchodilation. In some embodiments, the active ingredient comprises a cardiac stimulant or an inotropic agent, such as an adrenergic agonist. In some embodiments, the active ingredient comprises epinephrine.

### Calcitonin

In some embodiments, the active ingredient comprises a hormone. The hormone may be recombinantly synthesized (e.g., insulin) or purified from natural sources (e.g., calcitonin salmon). In some embodiments, the hormone may be used for treating a bone-related disorder, such as, for example, lower back pain, osteoporosis, or menopause. In some embodiments, the active ingredient comprises calcitonin salmon, or a pharmaceutically acceptable salt thereof.

### Cyclosporine A

In some embodiments, the active ingredient comprises an immune suppressant. In some embodiments, the immune suppressant is for treating an autoimmune disorder, such as, psoriasis, rheumatoid arthritis, or Crohn's disease. In some embodiments, the immune suppressant is for treating a rejection post organ transplant. In some embodiments, the immune suppressant comprises a calcineurin inhibitor, a corticosteroid, or an anti-metabolite. In some embodiments, the immune suppressant comprises a cyclosporine A.

In some embodiments, the pharmaceutical compositions provided herein comprises at least: about 0.05 mg, about 0.1 mg, about 0.15 mg, about 0.2 mg, about 0.25 mg, about 0.3 mg, about 0.4 mg, about 0.5 mg, about 0.6 mg, about 0.7 mg, about 0.8 mg, about 0.9 mg, about 1 mg, about 2.5 mg, about 5 mg, about 10 mg, about 15 mg, about 20 mg, about 25 mg, about 30 mg or more of the active ingredient or a pharmaceutically acceptable salt thereof. In some embodiments, the pharmaceutical compositions provided herein comprises at most: about 0.1 mg, about 0.15 mg, about 0.2 mg, about 0.25 mg, about 0.3 mg, about 0.4 mg, about 0.5 mg, about 0.6 mg, about 0.7 mg, about 0.8 mg, about 0.9 mg, about 1 mg, about 2.5 mg, about 5 mg, about 10 mg, about 15 mg, about 20 mg, about 25 mg, or about 30 mg of the active ingredient or a pharmaceutically acceptable salt thereof.

In some embodiments, the pharmaceutical compositions provided herein comprises from about 0.05 mg to about 50 mg of the active ingredient or a pharmaceutically acceptable salt thereof. In some embodiments, the pharmaceutical compositions provided herein comprises from about 0.05 mg to about 40 mg, from about 0.05 mg to about 30 mg, from about 0.1 mg to about 40 mg, from about 0.1 mg to about 30 mg, from about 0.1 mg to about 20 mg, from about 0.1 mg to about 10 mg, from about 0.5 mg to about 40 mg, from about 0.5 mg to about 30 mg, from about 0.5 mg to about 20 mg, from about 0.5 mg to about 10 mg, from about 1 mg to about 30 mg, from about 1 mg to about 25 mg, from about 1 mg to about 20 mg, from about 1 mg to about 15 mg, from about 1 mg to about 10 mg, from about 1 mg to about 9 mg, from about 1 mg to about 8 mg, from about 1 mg to about 7 mg, from about 1 mg to about 6 mg, from about 1 mg to about 5 mg, from about 2 mg to about 10 mg, from about 2 mg to about 9 mg, from about 2 mg to about 8 mg, from about 2 mg to about 7 mg, from about 2 mg to about 6 mg, or from about 2 mg to about 5 mg of the active ingredient or a pharmaceutically acceptable salt thereof.

In some embodiments, a total weight of the pharmaceutical compositions comprises at least: about 0.05%, about 0.1%, about 0.15%, about 0.2%, 0.25%, about 0.3%, about 0.35%, about 0.4%, about 0.45%, about 0.5%, about 0.6%, about 0.7%, about 0.8%, about 0.9%, about 1%, about 2%, about 3%, about 4%, about 5%, about 7.5%, about 10%, about 15%, about 20%, about 25%, about 30%, about 35%, about 40%, about 45%, or about 50% or more of the active ingredient or a pharmaceutically acceptable salt thereof. In some embodiments, a total weight of the pharmaceutical compositions comprises at most: about 0.5%, about 0.6%, about 0.7%, about 0.8%, about 0.9%, about 1%, about 5%, about 7.5%, about 10%, about 15%, about 20%, about 25%, about 30%, about 35%, about 40%, about 45%, or about 50% of the active ingredient or a pharmaceutically acceptable salt thereof.

In some embodiments, a total weight of the pharmaceutical compositions comprises from about 1% to about 60%, from about 1% to about 50%, from about 1% to about 40%, from about 1% to about 30%, from about 2% to about 50%, from about 2% to about 40%, from about 2% to about 30%, from about 5% to about 50%, from about 5% to about 40%, from about 5% to about 30%, from about 7.5% to about 50%, from about 7.5% to about 40%, from about 7.5% to about 30%, from about 7.5% to about 20%, from about 7.5% to about 15%, from about 7.5% to about 10%, from about 10% to about 50%, from about 10% to about 40%, from about 10% to about 30%, from about 10% to about 20%, from about 20% to about 40%, or from about 20% to about 35% of the active ingredient or a pharmaceutically acceptable salt thereof. In some embodiments, a total weight of the pharmaceutical compositions comprises from about 0.05% to about 0.4%, from about 0.05% to about 0.35%, from about 0.05% to about 0.3%, from about 0.05% to about 0.25%, from about 0.05% to about 0.2%, from about 0.05% to about 0.15%, or from about 0.05% to about 0.1% of the active ingredient or a pharmaceutically acceptable salt thereof.

### Excipient

In some embodiments, the pharmaceutical composition provided herein comprises an excipient. In some embodiments, the pharmaceutical composition comprises microcrystalline cellulose (MCC). In some embodiments, the pharmaceutical composition comprises at least 1, 2, 3, 4, 5 or more excipients or a pharmaceutically acceptable salt thereof. In some embodiments, the pharmaceutical composition comprises at most 1, 2, 3, 4, or 5 excipients or a pharmaceutically acceptable salt thereof.

In some embodiments, the pharmaceutical composition comprises at least: about 5 mg, about 10 mg, about 15 mg, about 20 mg, about 25 mg, about 30 mg, about 40 mg, about 50 mg, about 60 mg, about 70 mg, about 80 mg, about 90 mg, or about 100 mg or more of the excipient or a pharmaceutically acceptable salt thereof. In some embodiments, the pharmaceutical composition comprises about 50 mg to about 90 mg of an excipient. In some embodiments, the pharmaceutical composition comprises about 60 mg to about 80 mg of an excipient (e.g., MCC). In some embodiments, the pharmaceutical composition comprises about 50 mg, about 60 mg, about 70 mg, or about 80 mg of an excipient.

In some embodiments, the pharmaceutical composition comprises from about 5 mg to about 100 mg, from about 5 mg to about 90 mg, from about 5 mg to about 80 mg, from about 5 mg to about 70 mg, from about 5 mg to about 60 mg, from about 5 mg to about 50 mg, from about 5 mg to about 40 mg, from about 5 mg to about 30 mg, from about 10 mg to about 100 mg, from about 10 mg to about 80 mg, from about 10 mg to about 60 mg, from about 10 mg to about 40 mg, from about 15 mg to about 80 mg, from about 15 mg to about 70 mg, from about 15 mg to about 60 mg, from about 15 mg to about 50 mg, or from about 15 mg to about 40 mg of the excipient or a pharmaceutically acceptable salt thereof.

In some embodiments, a total weight of the pharmaceutical compositions comprises at least about 30%, at least about 40%, at least about 50%, at least about 60%, at least about 70%, at least about 80%, at least about 90%, at least about 95% or more of the excipient or a pharmaceutically acceptable salt thereof. In some embodiments, a total weight of the pharmaceutical compositions comprises at most about 30%, at most about 40%, most about 50%, at most about 60%, most about 70%, at most about 80%, most about 90%, or at most about 95% of the excipient or a pharmaceutically acceptable salt thereof.

In some embodiments, a total weight of the pharmaceutical compositions comprises from about 30% to about 95%, from about 30% to 90%, from about 30% to about 80%, from about 30% to 70%, from about 30% to about 60%, from about 30% to 50%, from about 40% to about 95%, from about 40% to 90%, from about 40% to about 80%, from about 40% to 70%, from about 40% to about 60%, from about 40% to 50%, %, from about 50% to about 95%, from about 50% to 90%, from about 50% to about 80%, from about 50% to 70%, from about 50% to about 60%, from about 60% to about 95%, from about 60% to about 90%, from about 60% to about 80%, or from about 60% to 70% of the excipient or a pharmaceutically acceptable salt thereof. In some embodiments, the excipient (e.g., MCC) is about 40% to about 70% of the pharmaceutical composition. In some embodiments, the excipient is about 50% to about 60% of the pharmaceutical composition. In some embodiments, the excipient is about 50% of the pharmaceutical composition. In some embodiments, the excipient is about 55% of the pharmaceutical composition. In some embodiments, the excipient is about 60% of the pharmaceutical composition.

In some embodiments, the pharmaceutical composition comprises an excipient. In some embodiments, pharmaceutical composition comprises one excipient. In some embodiments, the pharmaceutical composition comprises one or more excipients. In some embodiments, the pharmaceutical composition comprises two or more excipients. In some embodiments, the pharmaceutical composition comprises three or more excipients. In some embodiments, the pharmaceutical composition comprises two excipients. In some embodiments, the pharmaceutical composition comprises three excipients.

In some embodiments, the excipient comprises a carbohydrate molecule or a pharmaceutically acceptable salt thereof. In some embodiments, the carbohydrate molecule comprises a polysaccharide, an oligosaccharide, or a disaccharide. In some embodiments, the carbohydrate molecule comprises chitosan, microcrystalline cellulose (MCC), croscarmellose, starch, or lactose. In some embodiments, the carbohydrate molecule comprises MCC. In some embodiments, the excipient comprises a sweetener. In some embodiments, the sweetener comprises a disaccharide, a monosaccharide, or a sugar alcohol. In some embodiments, the sweetener comprises a lactose. In some embodiments, the excipient comprises a binder, an anticaking agent, an emulsifier, an extender, or a bulking agent. In some embodiments, the binder, anticaking agent, emulsifier, extender, or bulking agent comprises MCC.

In some embodiments, the excipient comprises a phosphate salt. In some embodiments, the phosphate salt comprises a calcium phosphate. In some embodiments, calcium phosphate comprises tribasic calcium phosphate.

In some embodiments, the pharmaceutical composition comprises microcrystalline cellulose, chitosan, lactose, croscarmellose sodium, tribasic calcium phosphate, or any combination thereof.

In some embodiments, the pharmaceutical composition comprises microcrystalline cellulose (MCC). Acceptable MCC may comprise MCC obtained by decomposing cellulose materials such as pulp by either or both of acid and alkaline hydrolyses, then purifying the hydrolysate, and crushing or grinding it before, during, or after drying. MCC of a select average particle diameter size may be obtained, for example, via appropriate processing, e.g., via fine grinding using a high-speed rotary impact mill or air attrition mill as necessary, and size sorting. In some embodiments, MCC of the pharmaceutical composition presented herein comprises products available under the trade names of CEOLUS® PH-F20JP (e.g., mean particle size about 20-23 microns, bulk density about 0.23 g/cm³, repose angle not less than 60 degrees), CEOLUS® PH-301 (e.g., mean particle size about 50 microns, bulk density about 0.41 g/cm³, repose angle about 41 degrees), CEOLUS® PH-101 (e.g., mean particle size about 50 microns, bulk density about 0.29 g/cm3, repose angle about 45 degrees), CEOLUS® PH-102 (e.g., mean particle size about 90 microns, bulk density about 0.3 g/cm³, repose angle about 42 degrees), and CEOLUS® PH-302 (available from Asahi Kasei Corporation, e.g., mean particle size about 90 microns, bulk density about 0.43 g/cm³, repose angle about 38 degrees), and AVICEL® PH-105 (e.g., mean particle size about 20 microns, bulk density about 0.20-0.30 g/cm³), AVICEL® PH-101 (e.g., mean particle size about 50 microns, bulk density about 0.26-0.31 g/cm³), AVICEL® PH-102 (e.g., mean particle size about 100 microns, bulk density about 0.28-0.33 g/cm³), AVICEL® PH-301 (e.g., mean particle size about 50 microns, bulk density about 0.34-0.45 g/cm³), and AVICEL® PH-302 (available from FMC Biopolymer Corporation, e.g., mean particle size about 100 microns, bulk density about 0.35-0.46 g/cm³). In some embodiments, the pharmaceutical composition comprises CEOLUS® PH-F20JP and CEOLUS® PH-301. In some embodiments, the pharmaceutical composition comprising MCC demonstrates enhanced pharmacokinetic parameters compared with a corresponding pharmaceutical composition that comprises another excipient (e.g., lactose, chitosan).

Mean particle size of the microcrystalline cellulose (MCC) of the pharmaceutical composition may be determined using standard techniques, such as, for example, a laser-diffraction particle size distribution analyzer or sorting methods. In some embodiments, the MCC has a mean particle size of from about 10 μm to about 100 μm, from about 10 μm to about 80 μm, from about 10 μm to about 60 μm, from about 10 μm to about 40 μm, from about 15 μm to about 100 μm, from about 15 μm to about 80 μm, from about 15 μm to about 60 μm, from about 15 μm to about 40 μm, from about 15 μm to about 25 μm, from about 20 μm to about 100 μm, from about 20 μm to about 80 μm, from about 20 μm to about 60 μm, from about 20 μm to about 40 μm, from about 30 μm to about 100 μm, from about m to about 80 μm, from about 30 μm to about 60 μm, from about 40 μm to about 100 μm, from about 40 μm to about 80 μm, from about 40 μm to about 60 μm, from about 50 μm to about 100 μm, from about 50 μm to about 80 μm, from about 50 μm to about 70 μm, or from about 65 μm to about 75 μm.

In some embodiments, the microcrystalline cellulose (MCC) has a mean particle size of at least: about 10 μm, about 15 μm, about 20 μm, about 25 μm, about 30 μm, about 40 μm, about 50 μm, about 60 μm, about 65 μm, about 70 μm, about 75 μm, about 80 μm, about 90 μm, or about 100 μm or more. In some embodiments, the pharmaceutical composition comprises MCC having a mean particle size of at most: about 10 μm, about 15 μm, about 20 μm, about 25 μm, about 30 μm, about 40 μm, about 50 μm, about 60 μm, about 65 μm, about 70 μm, about 75 μm, about 80 μm, about 90 μm, or about 100 μm.

In some embodiments, a total weight of the pharmaceutical compositions comprises from about 30% to about 95%, from about 30% to 90%, from about 30% to about 80%, from about 30% to 70%, from about 30% to about 60%, from about 30% to 50%, from about 40% to about 95%, from about 40% to 90%, from about 40% to about 80%, from about 40% to 70%, from about 40% to about 60%, from about 40% to 50%, %, from about 50% to about 95%, from about 50% to 90%, from about 50% to about 80%, from about 50% to 70%, from about 50% to about 60%, from about 60% to about 95%, from about 60% to 90%, from about 60% to about 80%, from about 60% to 70%, from about 70% to 90%, from about 80% to 90%, from about 85% to 95%, or from about 90% to 95% of microcrystalline cellulose.

In some embodiments, a total weight of the pharmaceutical compositions comprises at least about 30%, at least about 40%, at least about 50%, at least about 60%, least about 70%, at least about 80%, at least about 90%, at least about 95% or more of microcrystalline cellulose (MCC). In some embodiments, a total weight of the pharmaceutical compositions comprises at most about 30%, at most about 40%, at most about 50%, at most about 60%, at most about 70%, at most about 80%, at most about 90%, or at most about 95% of MCC.

In some embodiments, the microcrystalline cellulose (MCC) has a bulk density of at least 0.1, at least 0.2, at least 0.3, at least 0.4, at least 0.5, at least 0.6, or at least 0.7 g/cm³. In some embodiments, the pharmaceutical composition comprises MCC having a bulk density of at most 0.1, at most 0.2, at most 0.3, at most 0.4, at most 0.5, at most 0.6, or at most 0.7 g/cm³. In some embodiments, the MCC has a bulk density of from about 0.1 to 0.5 g/cm³, from about 0.1 to 0.4 g/cm³, from about 0.1 to 0.3 g/cm³, from about 0.1 to 0.2 g/cm³, about 0.2 to 0.5 g/cm³, from about 0.2 to 0.4 g/cm³, from about 0.2 to 0.3 g/cm³, from about 0.3 to 0.5 g/cm³, from about 0.3 to 0.4 g/cm³, or from about 0.4 to 0.5 g/cm³.

In some embodiments, the pharmaceutical composition provided herein comprises a weight ratio of absorption enhancer (e.g., choline ester) to excipient (e.g., microcrystalline cellulose) of at least: about 5:1, about 2:1, 1:1, about 1:5, about 1:10, about 1:15, about 1:20, about 1:25, about 1:30, about 1:35, about 1:40, about 1:45, about 1:50, about 1:60, about 1:70, about 1:80, about 1:90, about 1:100. In some embodiments, the pharmaceutical composition provided herein comprises a weight ratio of absorption enhancer (e.g., choline ester) to excipient (e.g., microcrystalline cellulose) of at most: about 5:1, about 2:1, 1:1, about 1:5, about 1:10, about 1:15, about 1:20, about 1:25, about 1:30, about 1:35, about 1:40, about 1:45, about 1:50, about 1:60, about 1:70, about 1:80, about 1:90, about 1:100. In some embodiments, the absorption enhancer and the excipient (e.g., MCC) are present in the pharmaceutical composition in a ratio of about 1:10 (absorption enhancer:excipient). In some embodiments, the absorption enhancer and the excipient (e.g., MCC) are present in the pharmaceutical composition in a ratio of about 1:13 (absorption enhancer:excipient). In some embodiments, the absorption enhancer and the excipient (e.g., MCC) are present in the pharmaceutical composition in a ratio of about 1:15 (absorption enhancer:excipient).

In some embodiments, the pharmaceutical composition provided herein comprises a weight ratio of absorption enhancer (e.g., choline ester) to excipient (e.g., microcrystalline cellulose) of from about 1:1 to about 1:100, from about 1:1 to about 1:90, from about 1:1 to about 1:80, from about 1:1 to about 1:70, from about 1:1 to about 1:60, from about 1:1 to about 1:50, from about 1:1 to about 1:40, from about 1:1 to about 1:30, from about 1:1 to about 1:20, from about 1:1 to about 1:10, from about 1:2 to about 1:50, from about 1:2 to about 1:40, from about 1:2 to about 1:30, from about 1:2 to about 1:20, from about 1:2 to about 1:10, from about 1:3 to about 1:50, from about 1:3 to about 1:40, from about 1:3 to about 1:30, from about 1:3 to about 1:20, from about 1:3 to about 1:10, from about 1:3 to about 1:5, from about 1:5 to about 1:50, from about 1:5 to about 1:40, from about 1:5 to about 1:50, from about 1:5 to about 1:20, from about 1:5 to about 1:10, 1:10 to about 1:100, from about 1:10 to about 1:90, from about 1:10 to about 1:80, from about 1:10 to about 1:70, from about 1:10 to about 1:60, from about 1:10 to about 1:50, from about 1:20 to about 1:90, from about 1:20 to about 1:80, from about 1:20 to about 1:70, from about 1:20 to about 1:60, or about 1:20 to about 1:50, from about 1:30 to about 1:90, from about 1:30 to about 1:80, from about 1:30 to about 1:70, from about 1:30 to about 1:60, or from about 1:30 to about 1:50.

In some embodiments, the pharmaceutical composition provided herein further comprises one or more excipient. In some embodiments, the one or more excipients comprise croscarmellose sodium (CCS), tribasic calcium phosphate (TCP), or a combination thereof.

### Pharmaceutical Composition

Some aspects of the present disclosure provide a pharmaceutical composition with improved mucosal penetration and/or absorption of active ingredients as compared to a corresponding pharmaceutical composition without an absorption enhancer. The pharmaceutical composition may have improved mucosal penetration and/or absorption of the active ingredient via a plurality of mucosal membranes, such as, for example, nasal mucosal membranes, buccal mucosal membranes, sublingual mucosal membranes, rectal mucosal membranes, oracular mucosal membranes, pulmonary mucosal membranes, vaginal mucosal membranes, or a combination thereof. In some embodiments, the pharmaceutical composition improves penetration and/or absorption of the active ingredient through the nasal mucus membrane.

In some embodiments, the pharmaceutical composition improves mucosal penetration and/or absorption of active ingredients compared to a corresponding pharmaceutical composition that does not comprise a choline ester (e.g., lauroylcholine chloride (LCC)). In some embodiments, the pharmaceutical composition improves mucosal penetration and/or absorption of active ingredients compared to a corresponding pharmaceutical composition that comprises the active ingredient and does not comprise an excipient (e.g., microcrystalline cellulose (MCC)). In some embodiments, the pharmaceutical composition comprising an active ingredient, a choline ester (e.g., LCC), and an excipient (e.g., MCC) demonstrates enhanced pharmacokinetic parameters (e.g., higher Cmax, higher AUC). In some embodiments, the pharmaceutical composition comprising an active ingredient, a choline ester (e.g., LCC), and MCC demonstrates enhanced pharmacokinetic parameters compared with a corresponding pharmaceutical composition that comprises another class of absorption enhancer (e.g., surfactant or sugar). In some embodiments, the pharmaceutical composition comprising an active ingredient, a choline ester (e.g., LCC), and MCC demonstrates enhanced pharmacokinetic parameters compared with a corresponding pharmaceutical composition that comprises another excipient (e.g., lactose, chitosan). In some embodiments, the pharmaceutical composition provided herein demonstrates enhanced pharmacokinetic parameters compared with a liquid pharmaceutical composition comprising an absorption enhancer (e.g., LCC).

The improved mucosal penetration and/or absorption of the active ingredient may be measured by numerous mechanisms, such as through pharmacokinetic (PK) parameters. The PK parameter may be any one or more of a plurality of PK parameters, such as a drug concentration (C), Cmax, Cmin, Tmax, half-life (t½), bioavailability (F), or area under curve (AUC). Bioavailability may be calculated based on the dose of the pharmaceutical composition administered and area under curve. The PK parameters may be obtained at any time after the pharmaceutical composition is administered. In some embodiments, the drug concentration, bioavailability and AUC are obtained at least: about 5 minutes, about 15 minutes, about 30 minutes, about 45 minutes, about 60 minutes, about 90 minutes, about 120 minutes, about 180 minutes, about 4 hours, about 6 hours, about 12 hours, about 24 hours, about 36 hours, about 48 hours, about 60 hours, about 72 hours, about 4 days, about 5 days, about 6 days, about 1 week, about 2 weeks, about 3 weeks, or about 4 weeks after the pharmaceutical composition is administered. Subscripts may be used to designate the time of PK measurement. For example, C_{30 min}, F_{0-30 min}, and AUC_{0-30 min} are drug concentration, bioavailability and AUC, respectively, obtained at about 30 minutes after the pharmaceutical composition is administered.

In some embodiments, the PK parameters are measured from a blood or plasma sample of a subject. In some embodiments, the PK parameters are measured from a non-blood sample of a subject, such as amniotic fluid, aqueous humor, bile, breast milk, lymph, mucus, urine, or cerebral spinal fluid. In some embodiments, the PK parameters are measured in a target tissue, such as a cerebral spinal fluid (CSF) or a brain parenchyma of a subject. In some embodiments, the PK parameters (e.g., drug concentration) are measured by liquid chromatography (LC), mass spectrometry (MS), Enzyme Immunoassay (EIA), enzyme-linked immunosorbent assay (ELISA), or any combination thereof (e.g., LC/MS/MS).

In some embodiments, the pharmaceutical composition (e.g., comprising an active ingredient and a choline ester) provides (e.g., to the subject having received the pharmaceutical composition) an improved pharmacokinetic (PK) parameter of the active agent compared to a corresponding pharmaceutical composition (e.g., containing the same active ingredient) that does not comprise a choline ester (e.g., LCC). An improved PK parameter may comprise a higher bioavailability, a larger AUC, a longer half-life, a higher Cmax, or a shorter Tmax, or any combination thereof. In some embodiments, the pharmaceutical composition (e.g., comprising an active ingredient and an excipient) provides an improved pharmacokinetic (PK) parameter of the active agent comprising a higher bioavailability, a larger AUC, a longer half-life, a higher Cmax, or a shorter Tmax, or any combination thereof, compared to a pharmaceutical composition that does not comprise an excipient (e.g., MCC). In some embodiments, the pharmaceutical composition comprising an active ingredient, excipient, and an absorption enhancer provides an improved pharmacokinetic (PK) parameter of the active agent comprising a higher bioavailability, a larger AUC, a longer half-life, a higher Cmax, or a shorter Tmax, or any combination thereof, compared to a pharmaceutical composition that does not comprise an excipient (e.g., MCC) or a choline ester (e.g., LCC).

In some embodiments, the pharmaceutical composition (e.g., comprising an active ingredient and an absorption enhancer) improves a PK parameter of an active agent by at least: about 5%, about 10%, about 15%, about 20%, about 30%, about 40%, about 50%, about 60%, about 70%, about 80%, about 90%, about 100%, about 150%, about 200%, about 250%, about 300%, about 350%, about 400%, about 450%, about 500%, about 600%, about 700%, about 800%, about 900%, about 1000%, about 1100%, about 1200%, about 1300%, about 1400%, about 1500%, or about 2000% compared to a pharmaceutical composition (e.g. comprising the same active ingredient) that does not comprise the choline ester (e.g., LCC).

In some embodiments, the improved PK parameter comprises an AUC parameter. In some embodiments, the improved AUC parameter comprises AUC_{0-10 min}, AUC_{0-30 min}, AUC_{0-60 min}, AUC_{0-90 min}, AUC_{0-120 min}, AUC_{0-150 min}, AUC_{0-240 min}, AUC_{0-300 min}, AUC_{0-480 min}, or any combination thereof. In some embodiments, the improved AUC parameter comprises AUC_{0-10 min}. In some embodiments, the improved AUC parameter comprises AUC_{0-30 min}. In some embodiments, the improved AUC parameter comprises AUC_{0-60 min}. In some embodiments, the improved AUC parameter comprises AUC_{0-90 min}. In some embodiments, the improved AUC parameter comprises AUC_{0-120 min}. In some embodiments, the improved AUC parameter comprises AUC_{0-150 min}. In some embodiments, the improved AUC parameter comprises AUC_{0-240 min}. In some embodiments, the improved AUC parameter comprises AUC_{0-300 min}. In some embodiments, the improved AUC parameter comprises AUC_{0-480 min}.

In some embodiments, the pharmaceutical composition comprises an AUC_{0-30 min} that improves by at least 100%, 200%, 300%, 400%, 500%, 600%, 700%, 800%, 900%, 1000%, 1100%, or 1200% compared to a corresponding pharmaceutical composition that does not comprise an absorption enhancer (e.g., LCC). In some embodiments, the pharmaceutical composition comprises an AUC_{0-60 min} that improves by at least 100%, 200%, 300%, 400%, 500%, 600%, 700%, 800%, 900%, or 1000% compared to a corresponding pharmaceutical composition that does not comprise the choline ester (e.g., LCC).

In some embodiments, the pharmaceutical composition has an improved AUC measured at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 90 minutes, at least 120 minutes, at least 2 hours, at least 3 hours, at least 6 hours, at least 8 hours, at least 10 hours, at least 12 hours, at least 15 hours, at least 24 hours, at least 36 hours, at least 48 hours, at least 3 days, at least 4 days, at least 5 days, at least 6 days, at least 7 days, at least 10 days, at least 2 weeks or more after the pharmaceutical composition is administered to a subject.

In some embodiments, the pharmaceutical composition comprises epinephrine or a pharmaceutically acceptable salt thereof, lauroylcholine chloride, and microcrystalline cellulose. In some embodiments, the pharmaceutical composition comprises a) about 1-5 mg of epinephrine or a pharmaceutically acceptable salt thereof; b) about 2-6 mg of lauroylcholine chloride; and c) about 51-55 mg of microcrystalline cellulose; wherein the pharmaceutical composition is formulated in a solid dosage form. In some embodiments, the pharmaceutical composition comprises a) about 3 mg of epinephrine or a pharmaceutically acceptable salt thereof, b) about 4 mg of lauroylcholine chloride; and c) about 53 mg of microcrystalline cellulose; wherein the pharmaceutical composition is formulated in a solid dosage form. In some embodiments, when the pharmaceutical composition is administered, a Cmax of epinephrine improves by at least about: 300%, 400%, 500%, 600%, 700%, or 800% compared to a corresponding pharmaceutical composition that comprises epinephrine and does not comprise lauroylcholine chloride. In some embodiments, when the pharmaceutical composition is administered, an AUC_{0-30 min} of epinephrine improves by at least about: 500%, 600%, 700%, 800%, 900%, 1000%, 1100%, or 1200% compared to a corresponding pharmaceutical composition that comprises epinephrine and does not comprise lauroylcholine chloride.

In some embodiments, the pharmaceutical composition comprises baclofen or a pharmaceutically acceptable salt thereof, lauroylcholine chloride, and microcrystalline cellulose. In some embodiments, the pharmaceutical composition comprises a) about 3-7 mg of baclofen or a pharmaceutically acceptable salt thereof; b) about 2-6 mg of lauroylcholine chloride; and c) about 49-53 mg of microcrystalline cellulose; wherein the pharmaceutical composition is formulated in a solid dosage form. In some embodiments, the pharmaceutical composition comprises a) about 5 mg of baclofen or a pharmaceutically acceptable salt thereof; b) about 4 mg of lauroylcholine chloride; and c) about 51 mg of microcrystalline cellulose; wherein the pharmaceutical composition is formulated in a solid dosage form. In some embodiments, when the pharmaceutical composition is administered, a Cmax of baclofen improves by at least about: 300%, 400%, or 500% compared to a corresponding pharmaceutical composition that comprises baclofen and does not comprise lauroylcholine chloride. In some embodiments, when the pharmaceutical composition is administered, an AUC_{0-30 min} of baclofen improves by at least about: 300%, 400%, or 500% compared to a corresponding pharmaceutical composition that comprises baclofen and does not comprise lauroylcholine chloride.

In some embodiments, the pharmaceutical composition comprises levodopa or a pharmaceutically acceptable salt thereof, lauroylcholine chloride, microcrystalline cellulose, and croscarmellose sodium. In some embodiments, the pharmaceutical composition comprises a) about 18-28 mg of levodopa or a pharmaceutically acceptable salt thereof, b) about 2-6 mg of LCC; c) about 25-50 mg of MCC; and, optionally, d) about 0.5-3 mg of croscarmellose sodium; wherein the pharmaceutical composition is formulated in a solid dosage form. In some embodiments, the pharmaceutical composition comprises a) about 18-22 mg of levodopa or a pharmaceutically acceptable salt thereof, b) about 2-6 mg of LCC; c) about 22-28 mg of MCC; and optionally d) about 1-2 mg of croscarmellose sodium; wherein the pharmaceutical composition is formulated in a solid dosage form. In some embodiments, the pharmaceutical composition comprises a) about 20 mg of levodopa or a pharmaceutically acceptable salt thereof; b) about 4 mg of LCC; c) about 34.2 mg of MCC; and d) about 1.8 mg of croscarmellose sodium; wherein the pharmaceutical composition is formulated in a solid dosage form. In some embodiments, the pharmaceutical composition comprises a) about 26.7 mg of levodopa or a pharmaceutically acceptable salt thereof; b) about 5.3 mg of LCC; c) about 45.6 mg of MCC; and d) about 2.4 mg of croscarmellose sodium; wherein the pharmaceutical composition is formulated in a solid dosage form. In some embodiments, the pharmaceutical composition comprises a) about 20 mg of levodopa or a pharmaceutically acceptable salt thereof, b) about 4 mg of LCC; and c) about 36 mg of MCC; wherein the pharmaceutical composition is formulated in a solid dosage form. In some embodiments, the pharmaceutical composition comprises a) about 26.7 mg of levodopa or a pharmaceutically acceptable salt thereof; b) about 5.3 mg of LCC; and c) about 48 mg of MCC; wherein the pharmaceutical composition is formulated in a solid dosage form. In some embodiments, when the pharmaceutical composition is administered, a Cmax of levodopa improves by at least about: 300%, 400%, 500%, 600%, or 700% compared to a corresponding pharmaceutical composition that does not comprise LCC. In some embodiments, when the pharmaceutical composition is administered, an AUC_{0-30 min} of levodopa improves by at least about: 300%, 400%, 500%, 600%, or 700% compared to a corresponding pharmaceutical composition that does not comprise LCC. In some embodiments, the pharmaceutical composition comprises about 20 mg to about 100 mg of the active ingredient (e.g., levodopa). In some embodiments, the pharmaceutical composition comprises about 10 mg to about 100 mg of the active ingredient (e.g., levodopa). In some embodiments, the pharmaceutical composition comprises about 60 mg to about 80 mg of the active ingredient (e.g., levodopa). In some embodiments, the pharmaceutical composition comprises about 20 mg to about 40 mg of the active ingredient (e.g., levodopa). In some embodiments, the pharmaceutical composition comprises about 20 mg to about 30 mg of the active ingredient (e.g., levodopa). In some embodiments, the pharmaceutical composition comprises about 20 mg of the active ingredient (e.g., levodopa). In some embodiments, the pharmaceutical composition comprises about 30 mg of the active ingredient (e.g., levodopa).

In some embodiments, the pharmaceutical composition comprises calcitonin or a pharmaceutically acceptable salt thereof, lauroylcholine chloride, and microcrystalline cellulose. In some embodiments, the pharmaceutical composition comprises a) about 0.1-0.2 mg of calcitonin or a pharmaceutically acceptable salt thereof; b) about 2-6 mg of lauroylcholine chloride; and c) about 54-57 mg of microcrystalline cellulose; wherein the pharmaceutical composition is formulated in a solid dosage form. In some embodiments, the pharmaceutical composition comprises a) about 0.12 mg of calcitonin or a pharmaceutically acceptable salt thereof, b) about 4 mg of lauroylcholine chloride; and c) about 55.88 mg of microcrystalline cellulose; wherein the pharmaceutical composition is formulated in a solid dosage form. In some embodiments, when the pharmaceutical composition is administered, a Cmax of calcitonin improves by at least about: 200%, 300%, or 400% compared to a corresponding pharmaceutical composition that comprises calcitonin and does not comprise lauroylcholine chloride. In some embodiments, when the pharmaceutical composition is administered, an AUC_{0-30 min} of calcitonin improves by at least about: 100%, 200%, or 300%, compared to a corresponding pharmaceutical composition that comprises calcitonin and does not comprise lauroylcholine chloride.

In some embodiments, the pharmaceutical composition comprises cyclosporine A or a pharmaceutically acceptable salt thereof, lauroylcholine chloride, and microcrystalline cellulose. In some embodiments, the pharmaceutical composition comprises a) about 28-32 mg of cyclosporine A or a pharmaceutically acceptable salt thereof, b) about 2-6 mg of lauroylcholine chloride; and c) about 24-28 mg of microcrystalline cellulose; wherein the pharmaceutical composition is formulated in a solid dosage form. In some embodiments, the pharmaceutical composition comprises a) about 30 mg of cyclosporine A or a pharmaceutically acceptable salt thereof, b) about 4 mg of lauroylcholine chloride; and c) about 26 mg of microcrystalline cellulose; wherein the pharmaceutical composition is formulated in a solid dosage form. In some embodiments, when the pharmaceutical composition is administered, an AUC_{0-30 min} of cyclosporine A improves by at least about: 400%, 500%, 600%, 700%, or 800%, compared to a corresponding pharmaceutical composition that comprises cyclosporine A and does not comprise lauroylcholine chloride.

In some embodiments, the pharmaceutical composition comprises a) a nucleic acid molecule or a pharmaceutically acceptable salt thereof, and b) an absorption enhancer comprising a choline ester; wherein the pharmaceutical composition is formulated in a solid dosage form. In some embodiments, the choline ester comprises a palmitoylcholine (PC) or a pharmaceutically acceptable salt thereof, a myristoylcholine (MyC) or a pharmaceutically acceptable salt thereof, or a lauroylcholine (LC) or a pharmaceutically acceptable salt thereof. In some embodiments, the pharmaceutical composition further comprising an excipient comprising a microcrystalline cellulose. In some embodiments, when the pharmaceutical composition is administered, a Cmax of the nucleic acid molecule improves by at least about: 50%, 80%, 100%, or 200% compared to a corresponding pharmaceutical composition that comprises the nucleic acid molecule and does not comprise an absorption enhancer. In some embodiments, when the pharmaceutical composition is administered, an AUC_{0-30 min} of the nucleic acid molecule improves by at least about: 100%, 200% or 300% compared to a corresponding composition that comprises the nucleic acid molecule and does not comprise an absorption enhancer.

In some embodiments, the pharmaceutical composition comprises a nucleic acid molecule or a pharmaceutically acceptable salt thereof, lauroylcholine, and microcrystalline cellulose. In some embodiments, the pharmaceutical composition comprises about 10 mg of the nucleic acid molecule or a pharmaceutically acceptable salt thereof, about 4 mg of lauroylcholine or a pharmaceutically acceptable salt thereof, and about 13 mg of microcrystalline cellulose. In some embodiments, the nucleic acid molecule comprises an antisense oligonucleotide.

In some embodiments, the pharmaceutical composition is for mucosal administration comprising buccal, sublingual, oral, ocular, intranasal, rectal, and intravaginal administration. In some embodiments, the pharmaceutical composition is for intranasal administration.

The pharmaceutical compositions provided herein are formulated in a solid dosage form comprising a powder, a tablet, a patch, or a capsule dosage form. In some embodiments, the pharmaceutical composition demonstrates enhanced pharmacokinetic parameters (e.g., higher Cmax, higher AUC) compared with a liquid dosage form. Solid dose forms may be characterized by physical properties (e.g., stability, solubility and dissolution rate) appropriate for pharmaceutical and therapeutic dosage forms. While not wishing to be bound by any particular theory, certain solid forms are characterized by physical properties (e.g., density, compressibility, hardness, morphology, cleavage, stickiness, solubility, water uptake, electrical properties, thermal behavior, solid-state reactivity, physical stability, and chemical stability) affecting particular processes (e.g., yield, filtration, washing, drying, milling, mixing, tableting, flowability, dissolution, formulation, and lyophilization) which make certain solid forms suitable for the manufacture of a solid dosage form. Such properties may be determined using particular analytical chemical techniques, including solid-state analytical techniques (e.g., X-ray diffraction, microscopy, spectroscopy and thermal analysis), as described herein and known in the art.

In some embodiments, a unit dose of the pharmaceutical composition comprises from about 20 mg to about 250 mg by weight. In some embodiments, a unit dose of the pharmaceutical composition comprises at least about 20 mg, at least about 25 mg, at least about 30 mg, at least about 40 mg, at least about 50 mg, at least about 100 mg, at least about 120 mg, at least about 150 mg, at least about 180 mg, at least about 200 mg, or at least about 225 mg or more by weight.

### Methods for Manufacturing

Some aspects of the present disclosure relate to methods for manufacturing the pharmaceutical composition. In some embodiments, the methods for manufacturing comprise blending, grinding, granulating, drying, or any combination thereof. In some embodiments, blending comprises mixing in a container (e.g., bag) or high-shear mixing in a granulator. In some embodiments, grinding comprises mortar grinding. In some embodiments, granulating comprises agitation granulating, extrusion granulating, fluid-bed granulating, high shear granulating, or a combination thereof. In some embodiments, granulating comprises passing the solid pharmaceutical composition through a sieve. In some embodiments, drying comprises freeze-drying, lyophilization, spray-drying, or spray freeze-drying.

### Methods for Treating

Some aspects of the present disclosure relate to a method for treating a disorder comprising administering to a subject in need thereof the pharmaceutical compositions provided herein. In some embodiments, the method comprises administering the pharmaceutical composition via buccal, sublingual, oral, ocular, intranasal, rectal, or intravaginal administration. In some embodiments, the preferred method comprises administering the pharmaceutical composition intranasally.

In some embodiments, the method provided herein comprises using the pharmaceutical compositions in diagnosing, preventing, or treating a disorder.

In some embodiments, the method comprises using the pharmaceutical compositions for treating a disorder of the central nervous system (CNS). In some embodiments, the disorder of the CNS comprises cerebral hemorrhage, cerebral infarction, CNS infections, brain tumor, Parkinson's disease, epilepsy, amyotrophic lateral sclerosis, Alzheimer's disease, Lewy body dementia, progressive supranuclear palsy, corticobasal degeneration, Pick's disease, frontotemporal dementia, multiple sclerosis, schizophrenia, depression, bipolar disorder, dysthymia, adjustment disorder, anxiety disorders, agitation, panic disorder, obsessive-compulsive disorder, autism spectrum disorder, attention deficit/hyperactivity disorder, sleep disorder, insomnia, traumatic brain injury, glioma, pain, and or migraine. In some embodiments, disorders of the CNS comprise tumors of the CNS comprising meningioma, pituitary adenoma, craniopharyngioma, Schwannoma, nasopharyngeal angiofibroma, choroid plexus tumors, dysembryoplastic neuroepithelial tumors, neurofibroma, hemangioblastoma, chondroma, giant cell tumor, osteoma, glioma (e.g., astrocytomas, oligodendrogliomas, or glioblastomas), ependymal tumors, hemangiopericytoma, germ cell tumors, or pineal tumors, chordoma, chondrosarcoma, medulloblastoma, olfactory neuroblastoma, lymphoma, gliosarcoma, rhabdomyosarcoma, paranasal sinus cancer, or atypical teratoid/rhabdoid tumor.

In some embodiments, the method comprises using the pharmaceutical compositions for treating a systemic disorder. In some embodiments, the systemic disorder comprises anaphylaxis, defervescence, pain, inflammation, rheumatism, hypnosis/sedation, anxiety, psychosis, depression, epilepsy, Parkinson's disease, cerebral circulatory metabolic disorder, muscle tone disorder, autonomic neuropathy, dizziness, migraine, hypertension, angina, arrhythmia, cardiovascular diseases, allergies, bronchodilation/asthma, respiratory diseases, peptic ulcer, gastrointestinal disorders, diarrhea, indigestion, gout, hyperuricemia, dyslipidemia, diabetes, hormonal disorders, pituitary hormone disorder, corticosteroid disorder, sex hormone disorder, uterine related diseases, osteoporosis, bone metabolism diseases, obesity, vitamin deficiency, malnutrition, poisoning, cancer, hyperimmunity, autoimmune disorders, otorhinolaryngology related diseases, mouth related diseases, urinary/genital diseases, hemorrhoids, skin diseases, hematopoiesis/blood coagulation related diseases, narcotic dependence, lifestyle related diseases.

In some embodiments, the method comprises using the pharmaceutical compositions for diagnosing a disorder. In some embodiments, the method comprises using the pharmaceutical compositions for a pre-operative or pre-examination treatment. In some embodiments, the pre-operative or pre-examination treatment comprises a radioactive agent for imaging.

In some embodiments, the method comprises using the pharmaceutical compositions for treating a disorder related to muscle spasticity. In some embodiments, the disorder related to muscle spasticity comprises multiple sclerosis, a spinal cord injury, dystonia, spasticity associated with cerebral palsy, trigeminal neuralgia, regional pain syndrome. In some embodiments, the method comprises using the pharmaceutical composition for treating a pain disorder. In some embodiments, the method comprises using the pharmaceutical composition for treating Parkinson's disease. In some embodiments, the method comprises using the pharmaceutical composition for treating postencephalitic parkinsonism, symptomatic parkinsonism, or motor fluctuations. In some embodiments, the method comprises using the pharmaceutical composition for treating a type I allergic reaction, such as anaphylaxis. In some embodiments, the method comprises using the pharmaceutical composition for treating hypotension associated with septic shock. In some embodiments, the method comprises using the pharmaceutical composition for treating a bone-related disorder, such as, for example, lower back pain, osteoporosis, or menopause. In some embodiments, the method comprises using the pharmaceutical composition for treating an autoimmune disorder, such as, psoriasis, rheumatoid arthritis, or Crohn's disease. In some embodiments, the method comprises using the pharmaceutical composition for treating a rejection post organ transplant, such as, grafts vs host disease.

In some aspects, provided herein includes a method of treating a disease or condition in a subject in need thereof, said method comprising administering a pharmaceutical composition to said subject, wherein said pharmaceutical composition comprises an active agent for treating Parkinson's disease, and an absorption enhancer comprising a choline ester. In some embodiments, the pharmaceutical composition further comprises microcrystalline cellulose. In some embodiments, the pharmaceutical composition is in a solid dosage form. The solid dosage form comprises a powder formulation for intranasal administration. In some embodiments, the active agent for treating Parkinson's disease comprises levodopa. In some embodiments, the administering produces a Tmax of less than 20 minutes. In some embodiments, the administering produces a Tmax of less than 15 minutes. In some embodiments, the administering produces a Tmax of less than 10 minutes. In some embodiments, the administering produces an AUC of greater than 10000 ng×min/mL. In some embodiments, the administering produces an AUC of greater than 12000 ng×min/mL. In some embodiments, the administering produces an AUC of greater than 15000 ng×min/mL. In some embodiments, the administering produces a Cmax of at least 300 ng/mL. In some embodiments, the administering produces a Cmax of at least 500 ng/mL.

In some embodiments, administering the pharmaceutical composition (e.g., comprising an active ingredient and an absorption enhancer) result in an improved pharmacokinetic (PK) parameter of the active agent compared to a corresponding pharmaceutical composition (e.g., containing the same active ingredient) that does not comprise an absorption enhancer (e.g., LCC). An improved PK parameter may comprise an increased bioavailability, an increased AUC, an increased half-life, an increased Cmax, or a shorter Tmax, or any combination thereof.

In some embodiments, administering the pharmaceutical composition provides an improved pharmacokinetic (PK) parameter of the active agent comprising an increased bioavailability, an increased AUC, an increased half-life, an increased Cmax, or a shorter Tmax, or any combination thereof, compared to a pharmaceutical composition that does not comprise an excipient (e.g., MCC). In some embodiments, the pharmaceutical composition comprising an active ingredient, excipient, and an absorption enhancer provides an improved pharmacokinetic (PK) parameter of the active agent comprising an increased bioavailability, an increased AUC, an increased half-life, an increased Cmax, or a shorter Tmax, or any combination thereof, compared to a pharmaceutical composition that does not comprise an excipient (e.g., MCC) or an absorption enhancer (e.g., LCC).

In some embodiments, administering the pharmaceutical composition result in an improved AUC parameter. In some embodiments, the improved AUC parameter comprises AUC_{0-10 min}, AUC_{0-20 min}, AUC_{0-30 min}, AUC_{0-60 min}, AUC_{0-90 min}, AUC_{0-120 min}, AUC_{0-150 min}, AUC_{0-240 min}, AUC₀₋₃₀₀ min, AUC_{0-480 min}, or any combination thereof. In some embodiments, the improved AUC parameter comprises AUC_{0-10 min}. In some embodiments, the improved AUC parameter comprises AUC_{0-20 min}. In some embodiments, the improved AUC parameter comprises AUC_{0-30 min}. In some embodiments, the improved AUC parameter comprises AUC_{0-60 min}. In some embodiments, the improved AUC parameter comprises AUC_{0-90 min}. In some embodiments, the improved AUC parameter comprises AUC_{0-120 min}. In some embodiments, the improved AUC parameter comprises AUC_{0-150 min}. In some embodiments, the improved AUC parameter comprises AUC_{0-240 min}. In some embodiments, the improved AUC parameter comprises AUC_{0-300 min}. In some embodiments, the improved AUC parameter comprises AUC_{0-480 min}.

In some embodiments, the pharmaceutical composition comprising a nucleic acid is administered intranasally as a solid formulation. In some embodiments, the pharmaceutical composition comprising a nucleic acid can be reconstituted in a pharmaceutically acceptable diluent and subsequently administered via an injection. In some embodiments, the injection is an intramuscular, a subcutaneous, an intravenous, or an intradermal injection.

In some embodiments, administering the pharmaceutical composition result in an AUC_{0-30 min} that improves by at least 100%, 200%, 300%, 400%, 500%, 600%, 700%, 800%, 900%, 1000%, 1100%, or 1200% compared to administering a corresponding pharmaceutical composition that does not comprise an absorption enhancer (e.g., LCC). In some embodiments, administering the pharmaceutical composition result in an AUC_{0-60 min} that improves by at least 100%, 200%, 300%, 400%, 500%, 600%, 700%, 800%, 900%, or 1000% compared to administering a corresponding pharmaceutical composition that does not comprise the absorption enhancer (e.g., LCC).

In some embodiments, administering the pharmaceutical composition result in an improved AUC measured at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 90 minutes, at least 120 minutes, at least 2 hours, at least 3 hours, at least 6 hours, at least 8 hours, at least 10 hours, at least 12 hours, at least 15 hours, at least 24 hours, at least 36 hours, at least 48 hours, at least 3 days, at least 4 days, at least 5 days, at least 6 days, at least 7 days, at least 10 days, at least 2 weeks or more after administering the pharmaceutical composition to a subject.

In some embodiments, the methods provided herein comprises administering an effective amount of the pharmaceutical composition. In some embodiments, the effective amount of the pharmaceutical composition comprises a lower dose of the active ingredient compared with a different formulation of the active ingredient. In some embodiments, the effective amount of the pharmaceutical composition comprises a dose that is at least 5%, 10%, 15%, 20%, 25%, 30%, 35%, 40%, 45%, or 50% or more lower than the dose of the active ingredient in a different formulation of the active ingredient.

In some embodiments, the methods provided herein comprises less toxicity. In some embodiments, the toxicity comprises a toxicity, a side effect, or an adverse drug event of the active ingredient.

The singular forms “a,” “an,” and “the” include plural referents unless the context clearly dictates otherwise. Thus, for example, reference to “a drug” includes reference to one or more of such drugs, and reference to “an excipient” includes reference to one or more of such excipients. When ranges are used herein, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range varies between 1% and 15% of the stated number or numerical range.

The terms “formulation” and “composition,” as used herein, are used interchangeably and refer to a mixture of two or more compounds, elements, or molecules. In some aspects, the terms “formulation” and “composition” may be used to refer to a mixture of one or more active agents with a carrier or other excipients.

The terms “active agent,” “active ingredient,” “active pharmaceutical agent,” “drug,” and variants thereof are used interchangeably to refer to an agent or substance that has measurable specified or selected physiologic activity when administered to a subject in a significant or effective amount. These terms are intended to encompass both parent drugs and prodrugs.

The terms “effective amount” or “therapeutically effective amount” as used herein, refer to a sufficient amount of an agent or a compound being administered which will relieve to some extent one or more of the symptoms of the disease or condition being treated. The result may be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined using techniques, such as a dose escalation study.

It is understood that substituents and substitution patterns on the compounds of the present invention may be selected by one of ordinary skilled person in the art to result chemically stable compounds which may be readily synthesized by techniques known in the art, as well as those methods set forth below, from readily available starting materials. If a substituent is itself substituted with more than one group, it is understood that these multiple groups may be on the same carbon or on different carbons, so long as a stable structure results. As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO-CEh-O-alkyl, -0P(0)(0-alkyl)2 or -CH2-0P(0)(0-alkyl)2. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent may be further substituted.

As used herein, an “absorption enhancer” is a compound that assist movement of active ingredients across epithelial barriers (e.g., in drug delivery). Absorption enhancers are functional excipients included in formulations to improve the absorption of an active ingredient. The term absorption enhancer usually refers to an agent whose function is to increase absorption by enhancing membrane permeation, rather than increasing solubility, so such agents are sometimes more specifically termed permeation enhancers.

As used herein, a “small molecule drug” refers to active pharmaceutical ingredients having a molecular weight of less than about 1000 grams/mole (g/mol, or Dalton/Da). In some embodiments, a small molecule drug has a molecular weight of less than about 500 Da. Small molecules drugs (e.g., insulin, aspirin, and antihistamines) make up about 90% of pharmaceutical drugs. Small molecule drugs also include biological molecules such as fatty acids, glucose, amino acids, and cholesterol and secondary metabolites such as lipids, glycosides, alkaloids, and natural phenols. Small molecules may comprise peptide drugs or non-peptide drugs. Small molecules do not include larger molecules such as polysaccharides, proteins, and nucleic acids.

As used herein, a “middle molecule drug” refers to active pharmaceutical ingredients having a molecular weight of from about 500 Da to about 50,000 Da. A middle molecule drug include biological molecules such as polysaccharides, proteins, and nucleic acids.

As used herein, the terms “ASO”, “antisense oligomer”, and “antisense oligonucleotide” are used interchangeably and refer to an oligomer such as a polynucleotide, comprising nucleobases that hybridize to a target nucleic acid (e.g., a pre-mRNA or an mRNA) sequence by Watson-Crick base pairing or wobble base pairing (G-U).

As used herein, the term “excipient” refers to an excipient suitable for use in a solid dosage form. The term “excipient” may be used interchangeably with “carrier”, when it refers to a water insoluble polysaccharide or oligosaccharide. The term “excipient” may be used interchangeably with “thickening agent”, when it refers to a water-soluble polysaccharide or oligosaccharide.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Manufacturing Powdered Compositions

The following materials were used in manufacturing the powdered formulations in the examples comprising active pharmaceutical ingredients, absorption enhancers, and excipients. Active Ingredients used include Epinephrine, Baclofen, Levodopa, Calcitonin Salmon, Cyclosporine A, and MALAT-1 ASO: 5′-CTAGTTCACTGAATGC-3′. Absorption enhancers used include lauroylcholine chloride (LCC), palmitoylcholine chloride, myristoylcholine chloride, butyrylcholine iodide, sodium laurylsulfate, and n-dodecyl-β-d-maltoside. excipients used include microcrystalline cellulose (e.g., CEOLUS (Registered Trademark) PH-F20JP and CEOLUS PH-301), chitosan, chitosan 11, lactose, respitose sv003, tribasic calcium phosphate, croscarmellose sodium, KICCOLATE (Registered Trademark) ND-200.

Physical properties of primary excipients were measured. The average primary particle diameter was measured under a dispersion pressure of 2 bar with a particle size distribution analyzer based on a laser diffraction method (Mastersizer 2000, Malvern Instrument Ltd.).

Based on the powder property measurement method of the Japanese Pharmacopoeia General Testing Method, the volume when each powder preparation having a known mass was placed in a graduated cylinder was measured, and the bulk density was calculated by dividing the mass by the volume.

The mean particle size and bulk density are shown in TABLE 1. MCC-1 is CEOLUS PH-F20JP; MCC-2 is CEOLUS PH-301. As shown in TABLE 1, the microcrystalline cellulose (MCC) used for this invention has a mean primary particle size of approximately 15 to 70 μm and a bulk density of approximately 0.25 to 0.45 g/cm³. The mean primary particle size of the MCC is similar to that of lactose. The bulk density of the MCC is similar to that of chitosan.

TABLE 1
Mean particle size and bulk density

| | Mean Particle | Bulk Density |
|---|---|---|
| | Size (Dv50, μm) | (g/cm³) |
| MCC-1 | 19.3 | 0.29 |
| MCC-2 | 66.8 | 0.44 |
| Chitosan | 94.2 | 0.31 |
| Lactose | 61.2 | 0.66 |

MCC-1 = CEOLUS PH-F20JP; MCC-2 = CEOLUS PH-301.

Powder compositions of the present disclosure may be manufactured by blending, grinding, agitation granulation, fluidized bed granulation, extruding granulation or lyophilization of components. As shown in TABLE 2, thirteen exemplary compositions (Example 1-13) and 21 control compositions (Comparative Examples 1-21) were manufactured with the following protocols.

For Examples 1-6 and 8-14, and Comparative (“Comp.”) Examples 7 to 9, 11, 17, 20, 22 and 23, the active ingredient, the absorption enhancer, and the excipient(s) were weighed according to the percentage of total weight (Weight %) of each component as listed in TABLE 2. The total weight of each composition was 10 g. To prepare a first-step mixture, the active ingredient, the absorption enhancer, and the excipient(s) were weighed and put into a plastic bag and preliminarily mixed. To prepare a second-step mixture, the first-step mixture was mixed at an agitator speed of 800 rpm and a chopper speed of 3000 rpm for 3 minutes using a mixing granulator (VGmm model, Powrex Corporation). To prepare a third-step mixture, the second-step mixture was further mixed while adding 3 to 7 mL of water over 1 minute and then mixed for additional 1 minute. To prepare a fourth-step mixture, the third-step mixture was removed from the granulator and was passed through a screen with a sieve opening of 1.7 mm (JIS Z 8801, Tokyo Screen Co., Ltd.) and then was dried at 50° C. for over 2 hours in a cryogenic thermostat (NDO-450SD, Tokyo Rikakikai Co., Ltd.). Finally, the fourth-step mixture was classified by powder formulation which remained on a screen with a sieve of 32 μm (JIS Z 8801, Iida-Seisakusho Japan Corporation) but passed through a screen with a sieve of 180 μm (JIS Z 8801, Iida-Seisakusho Japan Corporation).

For Example 7 and Comparative Example 24, the active ingredient, the absorption enhancer, and the excipient(s) listed in TABLE 2 were weighed according to the percentage of total weight of each component. The total weight of each composition was 120 mg. Powder formulation was prepared by blending each weighed component using a mortar for 5 minutes.

For Comparative Examples 1 and 3, the active ingredient and the absorption enhancer were weighed according to the weight percentage of total weight of each component, which is listed in TABLE 2. The total volume of each composition was 1 mL. The liquid formulation was prepared by dissolving each weighed component with 1 mL of pH 4.01 buffer solution using a vortex mixer and ultrasonic cleaning equipment.

For Comparative Examples 2, the active ingredient and the absorption enhancer were weighed according to the weight percentage of total weight of each component, which is listed in TABLE 2. The total volume of the composition was 10 mL. The liquid formulation was prepared by dissolving each weighed component with 10 mL of pH 4.01 buffer solution using a vortex mixer and ultrasonic cleaning equipment.

For Comparative Example 4 to 6, the active ingredient and the excipient(s) were weighed according to the percentage of total weight of each component, which is listed in TABLE 2. The total weight of each composition was 70 mg. Powder formulation was prepared by mixing each weighed component in a bottle using a vortex mixer for 1 minute.

For Comparative Example 10, the active ingredient and the absorption enhancer were weighed according to the percentage of total weight of each component, which is listed in TABLE 2. The total weight of the composition was 2 g. Powder formulation was prepared by mixing each weighed component in a bottle using a vortex mixer for 1 minute.

For Comparative Examples 12 to 14, the active ingredient and the absorption enhancer were weighed according to the weight percentage of total weight of each component, which is listed in TABLE 2. The total volume of 1 mL. Liquid formulation was prepared by dissolving each weighed component with 1 mL of saline using a vortex mixer and ultrasonic cleaning equipment.

For Comparative Example 15 and 16, the active ingredient and the absorption enhancer were weighed according to the percentage of total weight of each component, which is listed in TABLE 2 The total weight of each composition was 90 mg. Powder formulation was prepared by mixing each weighed component in a bottle using a vortex mixer for 1 minute.

For Comparative Examples 18 and 19, the active ingredient, the absorption enhancer and the excipient(s) were weighed according to the percentage of total weight of each component, which is listed in TABLE 2. The total weight of each composition was 1 g. Powder formulation was prepared by mixing each weighed component in a bottle using a vortex mixer for 1 minute.

For Comparative Example 21, the active ingredient, the absorption enhancer and the excipient(s) were weighed according to the percentage of total weight of each component, which is listed in TABLE 2. The total weight of each composition was 300 g. To prepare a first-step mixture, the active ingredient, the absorption enhancer, and the excipient(s) were weighed and put into a plastic bag and preliminarily mixed. To prepare a second-step mixture, the first-step mixture was mixed at an agitator speed of 400 rpm and a chopper speed of 1500 rpm for 1 minute using a high-speed mixing granulator (FS-GS-5 model, FUKAE Powtec Corporation). To prepare a third-step mixture, the second-step mixture was further mixed while adding 250 mL of water over 3 minute and then mixed for additional 1 minute. To prepare a fourth-step mixture, the third-step mixture removed from the granulator was dried at 50° C. for over 2 hours in a compartment tray dryer (NDO-450SD, Tokyo Rikakikai Co., Ltd.). Finally, the fourth-step mixture was classified by powder formulation which remained on a screen with a sieve of 32 μm (JIS Z 8801, Iida-Seisakusho Japan Corporation) but passed through a screen with a sieve of 180 μm (JIS Z 8801, Iida-Seisakusho Japan Corporation).

TABLE 2
Compositions of Powder Preparations

| | Delivered | | Active | | Absorption | | | | | | |
|---|---|---|---|---|---|---|---|---|---|---|---|
| Composition | dose | | Ingredient (%) | | Enhancer (%) | | Excipient (%) | | | | Form |
| Example 1 | 60 | mg | EPI | 5.0 | LCC | 6.7 | MCC-1 | 88.3 | — | — | Powder |
| Example 2 | 60 | mg | EPI | 5.0 | LCC | 6.7 | MCC-2 | 88.3 | — | — | Powder |
| Example 8 | 60 | mg | EPI | 5.0 | PCC | 6.7 | MCC-1 | 88.3 | — | — | Powder |
| Example 9 | 60 | mg | EPI | 5.0 | MyCC | 6.7 | MCC-1 | 88.3 | — | — | Powder |
| Comp. Example 1 | 100 | μl | EPI | 2.9 | — | — | — | — | — | — | Liquid |
| Comp. Example 2 | 100 | μl | EPI | 2.8 | LCC | 3.7 | — | — | — | — | Liquid |
| Comp. Example 3 | 100 | μl | EPI | 2.8 | SLS | 3.7 | — | — | — | — | Liquid |
| Comp. Example 4 | 7 | mg | EPI | 43.0 | LCC | 57.0 | — | — | — | — | Powder |
| Comp. Example 5 | 7 | mg | EPI | 43.0 | SLS | 57.0 | — | — | — | — | Powder |
| Comp. Example 6 | 7 | mg | EPI | 43.0 | DDM | 57.0 | — | — | — | — | Powder |
| Comp. Example 7 | 60 | mg | EPI | 5.0 | — | — | MCC-1 | 95.0 | — | — | Powder |
| Comp. Example 8 | 60 | mg | EPI | 5.0 | — | — | Chitosan | 95.0 | — | — | Powder |
| Comp. Example 9 | 60 | mg | EPI | 5.0 | LCC | 6.7 | Chitosan | 88.3 | — | — | Powder |
| Comp. Example 10 | 60 | mg | EPI | 5.0 | LCC | 6.7 | Lactose | 88.3 | — | — | Powder |
| Comp. Example 11 | 60 | mg | EPI | (5.0 | BCI | 6.2 | MCC-1 | 88.8 | — | — | Powder |
| Example 3 | 60 | mg | Baclofen | 8.3 | LCC | 6.7 | MCC-1 | 85.0 | — | — | Powder |
| Example 10 | 60 | mg | Baclofen | 8.3 | PCC | 6.7 | MCC-2 | 85.0 | — | — | Powder |
| Example 11 | 60 | mg | Baclofen | 8.3 | MyCC | 6.7 | MCC-1 | 85.0 | — | — | Powder |
| Comp. Example 12 | 100 | μl | Baclofen | 4.8 | — | — | — | — | — | — | Liquid |
| Comp. Example 13 | 100 | μl | Baclofen | 4.6 | LCC | 3.7 | — | — | — | — | Liquid |
| Comp. Example 14 | 100 | μl | Baclofen | 4.6 | SLS | 3.7 | — | — | — | — | Liquid |
| Comp. Example 15 | 9 | mg | Baclofen | 55.6 | SLS | 44.4 | — | — | — | — | Powder |
| Comp. Example 16 | 9 | mg | Baclofen | 55.6 | DDM | 44.4 | — | — | — | — | Powder |
| Comp. Example 17 | 60 | mg | Baclofen | 8.3 | — | — | MCC-1 | 91.7 | — | — | Powder |
| Comp. Example 18 | 60 | mg | Baclofen | 8.3 | — | — | Lactose | 91.7 | — | — | Powder |
| Comp. Example 19 | 60 | mg | Baclofen | 8.3 | LCC | 6.7 | Lactose | 85.0 | — | — | Powder |
| Comp. Example 20 | 60 | mg | Baclofen | 8.3 | SLS | 6.7 | MCC-1 | 85.0 | — | — | Powder |
| Example 4 | 50 | mg | LEVO | 40.0 | LCC | 8.0 | MCC-1 | 49.0 | CCS 3.0 | | Powder |
| Example 12 | 50 | mg | LEVO | 40.0 | PCC | 8.0 | MCC-1 | 52.0 | — | — | Powder |
| Example 13 | 50 | mg | LEVO | 40.0 | MyCC | 8.0 | MCC-1 | 52.0 | — | — | Powder |
| Example 14 | 50 | mg | LEVO | 40.0 | LCC | 8.0 | MCC-1 | 52.0 | — | — | Powder |
| Comp. Example 21 | 100 | mg | LEVO | 50.0 | — | — | MCC-1 | 50.0 | — | — | Powder |
| Comp. Example 23 | 60 | mg | Cyclo A | 50.0 | — | — | MCC-1 | 50.0 | — | — | Powder |
| Example 6 | 60 | mg | Cyclo | 50.0 | LCC | 6.7 | MCC-1 | 43.3 | — | — | Powder |
| Example 5 | 60 | mg | Calci | 0.2 | LCC | 6.7 | MCC-1 | 93.1 | — | — | Powder |
| Comp. Example 22 | 60 | mg | Calci | 0.2 | — | — | MCC-1 | 99.8 | — | — | Powder |
| Example 7 | 70 | mg | ASO | 33.3 | LCC | 13.3 | MCC-1 | 42.6 | MCC-2 10.0 | TCP 0.8 | Powder |
| Comp. Example 24 | 70 | mg | ASO | 33.3 | — | — | MCC-1 | 55.9 | MCC-2 10.0 | TCP 0.8 | Powder |

Abbreviations in TABLE 2: ASO=MALAT-1 antisense oligonucleotide; LCC=lauroylcholine chloride; PCC=palmitoylcholine chloride; MyCC, myristoylcholine chloride; EPI, epinephrine; LEVO, levodopa; Cyclo, cyclosporin; Calci, calcitonin; BCI, butyrylcholine iodide; SLS, sodium laurylsulfate; DDM, n-dodecyl-β-D-maltoside; MCC-1, CEOLUS PH-F20JP; MCC-2, CEOLUS PH-301; TCP, tribasic calcium phosphate; CCS, croscarmellose sodium.

### Example 2: Assessing Absorption of Epinephrine Intranasal Powder Compositions in Monkeys

TABLE 3 shows pharmacokinetic profiles of epinephrine compositions after intranasal administration. 60 mg of each Examples 1, 2, 8, and 9, Comparative Examples 7 to 11, and 7 mg of Comparative Example 4 to 6 were all delivered into the right nasal cavity of conscious male cynomolgus monkeys with an intranasal delivery device for powder formulation (at an equivalent dose of 3 mg epinephrine). The intranasal delivery device comprises a nozzle, a container for a unit dose of powder formulation, and an air generating unit (SNBL, Ltd.). 100 μL of each Comparative Examples 1-3 were delivered into the right nasal cavity of conscious male cynomolgus monkeys with a mucosal atomization device (MAD) (at an equivalent dose of 3 mg epinephrine).

TABLE 3
PK parameters of epinephrine after intranasal administration

| | | | | | |
|---|---|---|---|---|---|
| Time (min)/Plasma Concentration (ng/mL) | Cmax | Tmax | AUC₀₋₂₀ | AUC₀₋₃₀ | AUC₀₋₉₀ |

| | | | | | | | | | | | | |
|---|---|---|---|---|---|---|---|---|---|---|---|---|
| Composition | Subject | 0 | 2 | 5 | 10 | 20 | 30 | 60 | 90 | (ng/mL) | (min) | (ng · min/mL) |

| | | | | | | | | | | | | | | |
|---|---|---|---|---|---|---|---|---|---|---|---|---|---|---|
| Example 1 | 1 | 0.9 | 31.7 | 30.5 | 21.3 | 7.9 | 5.6 | 6.1 | 2.9 | 31.7 | 2.0 | 401.3 | 468.6 | 778.1 |
| | 2 | 1.2 | 13.8 | 24.9 | 24.1 | 19.8 | 20.4 | 9.4 | 6.4 | 24.9 | 5.0 | 415.0 | 615.9 | 1300.5 |
| | Mean | 1.0 | 22.7 | 27.7 | 22.7 | 13.8 | 13.0 | 7.7 | 4.6 | 28.3 | 3.5 | 408.1 | 542.3 | 1039.3 |
| Example 2 | 1 | 0.5 | 13.7 | 28.0 | 15.6 | 19.8 | 16.3 | 6.0 | 4.7 | 28.0 | 5.0 | 362.9 | 543.7 | 1039.1 |
| | 2 | 0.9 | 26.0 | 40.8 | 47.4 | 7.6 | 4.4 | 2.3 | 3.9 | 47.4 | 10.0 | 622.1 | 681.7 | 873.1 |
| | Mean | 0.7 | 19.8 | 34.4 | 31.5 | 13.7 | 10.4 | 4.1 | 4.3 | 37.7 | 7.5 | 492.5 | 612.7 | 956.1 |
| Example 8 | 1 | 1.1 | 3.2 | 5.4 | 8.0 | 12.3 | 10.6 | 3.1 | 1.9 | 12.3 | 20.0 | 152.3 | 266.9 | 547.2 |
| | 2 | 0.6 | 1.3 | 3.3 | 9.8 | 9.9 | 7.8 | 6.6 | 7.3 | 9.9 | 20.0 | 140.0 | 228.7 | 654.3 |
| | Mean | 0.9 | 2.3 | 4.3 | 8.9 | 11.1 | 9.2 | 4.9 | 4.6 | 11.1 | 20.0 | 146.1 | 247.8 | 600.7 |
| Example 9 | 1 | 2.6 | 7.6 | 14.1 | 21.1 | 21.7 | 19.9 | 15.4 | 8.0 | 21.7 | 20.0 | 344.6 | 552.1 | 1430.5 |
| | 2 | 0.9 | 10.5 | 24.1 | 19.4 | 16.5 | 19.2 | 10.6 | 9.2 | 24.1 | 5.0 | 351.3 | 529.9 | 1274.7 |
| | Mean | 1.7 | 9.0 | 19.1 | 20.3 | 19.1 | 19.5 | 13.0 | 8.6 | 22.9 | 12.5 | 347.9 | 541.0 | 1352.6 |

PK parameters of epinephrine after intranasal administration (Comparative Examples)

| | | Time (min)/Plasma Concentration (ng/mL) | | | | | | | | Cmax | Tmax | AUC₀₋₂₀ | AUC₀₋₃₀ | AUC₀₋₉₀ |
|---|---|---|---|---|---|---|---|---|---|---|---|---|---|---|
| Composition | Subject | 0 | 2 | 5 | 10 | 20 | 30 | 60 | 90 | (ng/mL) | (min) | (ng · min/mL) | | |
| Comp. Example 1 | 1 | 1.7 | 2.5 | 2.5 | 2.3 | 1.8 | 2.1 | 1.5 | 4.7 | 4.7 | 90.0 | 43.8 | 63.2 | 209.4 |
| | 2 | 0.5 | 0.6 | 3.1 | 0.7 | 0.4 | 0.3 | 0.5 | 0.7 | 3.1 | 5 | 21.5 | 25.1 | 56.6 |
| | Mean | 1.1 | 1.5 | 2.8 | 1.5 | 1.1 | 1.2 | 1.0 | 2.7 | 3.9 | 47.5 | 32.7 | 44.1 | 133.0 |
| Comp. Example 2 | 1 | 0.7 | — | 5.2 | 7.9 | 4.3 | 3.3 | 11.0 | 5.8 | 11.0 | 60.0 | 108.3 | 146.2 | 611.5 |
| | 2 | 0.5 | — | 5.1 | 8.4 | 5.0 | 6.8 | 5.1 | 5.0 | 8.4 | 10.0 | 114.8 | 174.0 | 505.0 |
| | Mean | 0.6 | — | 5.1 | 8.2 | 4.6 | 5.1 | 8.0 | 5.4 | 9.7 | 35.0 | 111.6 | 160.1 | 558.3 |
| Comp. Example 3 | 1 | 0.2 | 1.0 | 10.0 | 11.7 | 4.5 | 4.0 | 1.3 | 0.8 | 11.7 | 10.0 | 152.9 | 195.2 | 303.9 |
| | 2 | 1.0 | 2.3 | 1.9 | 1.9 | 1.1 | 0.8 | 1.1 | 1.7 | 2.3 | 2.0 | 34.0 | 43.8 | 116.3 |
| | Mean | 0.6 | 1.6 | 5.9 | 6.8 | 2.8 | 2.4 | 1.2 | 1.3 | 7.0 | 6.0 | 93.5 | 119.5 | 210.1 |
| Comp. Example 4 | 1 | 1.2 | — | 3.9 | 2.1 | 1.5 | 1.8 | 0.9 | 2.1 | 3.9 | 5 | 45.3 | 61.3 | 145.8 |
| | 2 | 1.9 | — | 1.2 | 0.6 | 0.7 | 0.5 | 0.5 | 0.6 | 1.9 | 0 | 18.8 | 25.0 | 57.6 |
| | Mean | 1.5 | — | 2.5 | 1.4 | 1.1 | 1.1 | 0.7 | 1.3 | 2.9 | 2.5 | 32 | 43 | 101.7 |
| Comp. Example 5 | 1 | 0.7 | 1.6 | 7.9 | 10.5 | 9.2 | 12.8 | 7.7 | 6.5 | 12.8 | 30.0 | 121.6 | 271.1 | 857.3 |
| | 2 | 0.7 | 2.2 | 3.1 | 3.1 | 3.0 | 3.9 | 3.6 | 3.1 | 3.9 | 30.0 | 51.8 | 91.4 | 299.2 |
| | Mean | 0.7 | 1.9 | 5.5 | 6.8 | 6.1 | 8.4 | 5.7 | 4.8 | 8.4 | 30.0 | 86.7 | 181.3 | 578.2 |
| Comp. Example 6 | 1 | 0.8 | 3.5 | 4.2 | 2.7 | 4.6 | 8.6 | 4.6 | 3.7 | 8.6 | 30 | 69.3 | 135.2 | 456.1 |
| | 2 | 0.3 | 7.4 | 2.8 | 1.6 | 3.5 | 1.5 | 3.0 | 1.6 | 7.4 | 2 | 59.6 | 84.7 | 221.2 |
| | Mean | 0.5 | 5.5 | 3.5 | 2.1 | 4.1 | 5.0 | 3.8 | 2.6 | 8.0 | 16 | 64.4 | 109.9 | 338.6 |
| Comp. Example 7 | 1 | 1.6 | — | 4.6 | 3.9 | 7.3 | 8.3 | 6.7 | 3.0 | 8.3 | 30 | 92.2 | 169.8 | 538.4 |
| | 2 | 1.2 | — | 1.5 | 1.8 | 2.4 | 3.5 | 3.8 | 4.0 | 4 | 90.0 | 36.0 | 65.1 | 291.6 |
| | Mean | 1.4 | — | 3.0 | 2.9 | 4.8 | 5.9 | 5.2 | 3.5 | 6.1 | 60 | 64.1 | 117.4 | 415.0 |
| Comp. Example 8 | 1 | 1.8 | — | 1.1 | 3.3 | 2.4 | 2.6 | 3.0 | 2.3 | 3.3 | 10.0 | 47.2 | 72.1 | 235.6 |
| | 2 | 1.5 | — | 1.4 | 3.1 | 2.5 | 1.6 | 1.7 | 0.8 | 3.1 | 10.0 | 46.2 | 66.5 | 152.4 |
| | Mean | 1.6 | — | 1.2 | 3.2 | 2.4 | 2.1 | 2.4 | 1.5 | 3.2 | 10.0 | 46.7 | 69.3 | 194.0 |
| Comp. Example 9 | 1 | 0.7 | — | 13.1 | 15.8 | 8.2 | 7.2 | 3.7 | 1.9 | 15.8 | 10.0 | 226.9 | 304.2 | 550.6 |
| | 2 | 0.8 | — | 3.9 | 4.0 | 5.4 | 2.9 | 2.6 | 3.5 | 5.4 | 20.0 | 78.1 | 119.2 | 292.0 |
| | Mean | 0.8 | — | 8.5 | 9.9 | 6.8 | 5.0 | 3.1 | 2.7 | 10.6 | 15.0 | 152.5 | 211.7 | 421.3 |
| Comp. Example 10 | 1 | 0.7 | — | 7.7 | 8.8 | 7.9 | 6.6 | 13.9 | 9.1 | 13.9 | 60.0 | 146.0 | 218.3 | 870.2 |
| | 2 | 1.8 | — | 9.3 | 9.6 | 6.4 | 4.8 | 5.2 | 3.4 | 9.6 | 10.0 | 154.6 | 210.3 | 488.8 |
| | Mean | 1.2 | — | 8.5 | 9.2 | 7.1 | 5.7 | 9.5 | 6.3 | 11.7 | 35.0 | 150.3 | 214.3 | 679.5 |
| Comp. Example 11 | 1 | 0.6 | — | 2.7 | 2.8 | 2.2 | 4.2 | 2.3 | 1.9 | 4.2 | 30.0 | 46.7 | 78.4 | 237.8 |
| | 2 | 1.5 | — | 3.0 | 6.4 | 5.3 | 4.4 | 2.7 | 1.3 | 6.4 | 10 | 93.5 | 142.2 | 307.2 |
| | Mean | 1.1 | — | 2.9 | 4.6 | 3.7 | 4.3 | 2.5 | 1.6 | 5.3 | 20 | 70.1 | 110.3 | 272.5 |

For measurement of the plasma epinephrine concentration, blood was collected from the femoral vein and collected into test tube with EDTA-2K. The plasma epinephrine concentrations were measured by HPLC method.

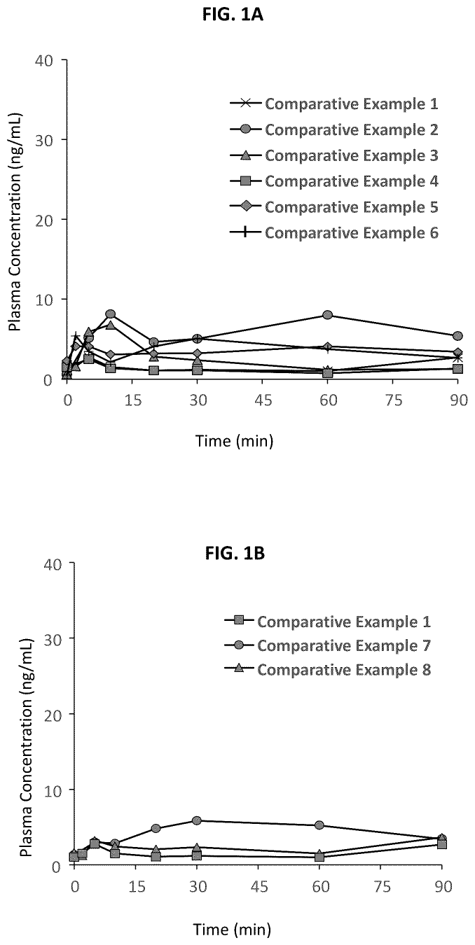

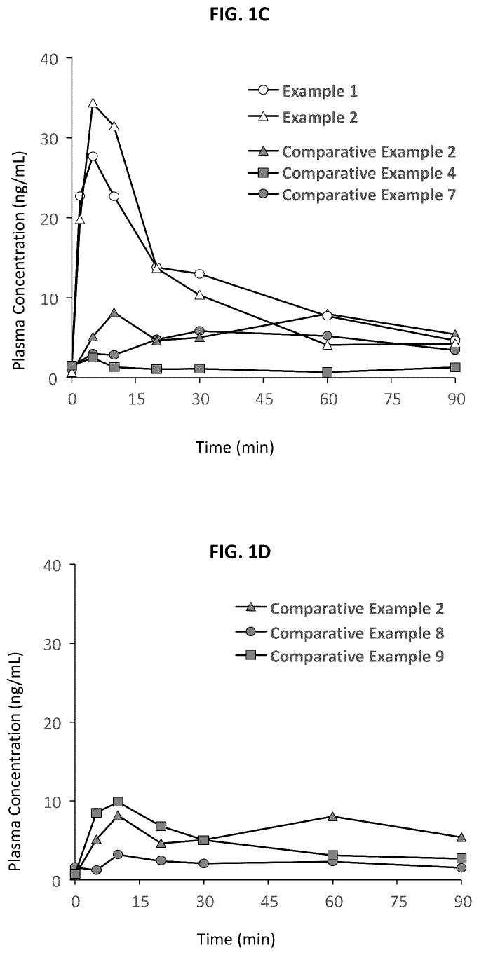

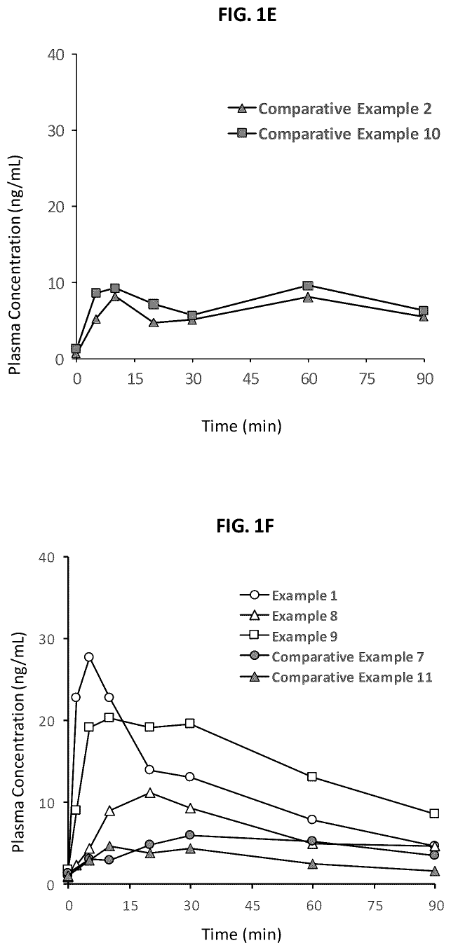

The plasma epinephrine concentration—time profiles, and the corresponding plasma epinephrine concentrations and PK parameters are shown in FIGS. 1A-1F and TABLE 3, respectively.

As shown in FIG. 1A, compositions comprising an absorption enhancer (e.g., LCC, SLS or DDM) in Comparative Examples 2-6, showed comparable or higher epinephrine concentrations compared with the composition without an absorption enhancer (Comparative Example 1). As shown in FIG. 1B, epinephrine compositions of Comparative Examples 7 and 8. comprise excipients (MCC or chitosan) without absorption enhancers. These compositions had comparable PK profiles compared with the epinephrine composition of Comparative Example 1. As shown in FIG. 1C, the compositions in Examples 1 and 2 have a combination of LCC and MCC, which drastically increased plasma epinephrine concentration compared to using LCC or MCC alone. There is a surprising synergistic effect on plasma concentration of epinephrine when LCC and MCC are combined in a composition. The improvement in the absorption of an epinephrine composition comprising LCC and MCC (e.g., Example 1 or 2) is much greater than the sum of improvement in absorption of epinephrine compositions comprising either LCC or MCC alone (Comparative Examples 2 and 7). FIG. 1D shows plasma epinephrine concentrations after administration of a liquid formulation comprising epinephrine and lauroylcholine chloride (LCC), a powder formulation comprising epinephrine and chitosan, and a powder formulation comprising epinephrine, LCC, and chitosan. FIG. 1E shows plasma epinephrine concentrations after administration of formulations comprising epinephrine and LCC or comprising epinephrine, LCC, and lactose. FIG. 1F shows plasma epinephrine concentrations after administration of formulations comprising: 1) epinephrine, a choline ester (e.g., LCC, PCC, or MyCC), and MCC; 2) epinephrine and MCC; or 3) epinephrine, butyrylcholine iodide (BCI), and MCC.

As shown in FIG. 1F, the composition in Comparative Example 11 having a combination of butyrylcholine (BCI) and MCC did not increase plasma epinephrine concentration, while the epinephrine compositions comprising both LCC and MCC showed the most improved absorption in this example.

AUC_{0-20 min} and AUC_{0-30 min} values indicate absorbability in an early period after dosing. As shown in TABLE 3, AUC_{0-20 min} and AUC_{0-30 min} of epinephrine compositions comprising both an alkanoylcholine (e.g., LCC) and MCC (e.g., Example 1 and Example 2) were drastically higher than AUC_{0-20 min} and AUC_{0-30 min} of compositions comprising MCC without any alkanoylcholine (Comparative Example 7). AUC_{0-30 min} of Example 1 was about 4.6 times the AUC_{0-30 min} of Comparative Example 7. AUC_{0-20 min} of Example 1 was about 6.4 times the AUC_{0-20 min} of Comparative Example 7. AUC_{0-30 min} of Example 2 was about 5.2 times the AUC_{0-30 min} of Comparative Example 7. AUC_{0-20 min} of Example 2 was about 7.7 times the AUC_{0-20 min} of Comparative Example 7. In some embodiments, LCC is the preferred alkanoylcholine absorption enhancer. Other PK parameters, such as Cmax and AUC observed at later time points (AUC_{0-60 min}, AUC_{0-90 min}), are much higher for the epinephrine compositions comprising both an alkanoylcholine (e.g., LCC) and MCC compared to other compositions. In summary, alkanoylcholines are great absorption enhancers for epinephrine when combined with MCC.

### Example 3: Assessing Absorption of Baclofen Intranasal Powder Compositions in Monkeys

TABLE 4 shows the pharmacokinetic profiles of baclofen after intranasal administration of various baclofen compositions. 60 mg of Example 3, 10, and 11, 60 mg of each Comparative Examples 17-20, and 9 mg of Comparative Example 15 and 16 were delivered into the right nostrils of conscious male cynomolgus monkeys with an intranasal delivery device for powder formulation (at an equivalent dose of 5 mg baclofen). The device is composed of a nozzle, a container for a unit dose of powder formulation and an air generating unit. 100 μL of each Comparative Examples 12-14 were delivered into the right nostrils of conscious male cynomolgus monkeys, with an MAD device (at an equivalent dose of 5 mg baclofen).

TABLE 4
Pharmacokinetic profiles of baclofen after intranasal administration

| | | Subject | | | | | | | | | Cmax | Tmax | AUC₀₋₂₀ | AUC₀₋₃₀ | AUC₀₋₂₄₀ |
|---|---|---|---|---|---|---|---|---|---|---|---|---|---|---|---|
| Composition | Subject | 0 | 5 | 10 | 20 | 30 | 60 | 90 | 120 | 240 | (ng/mL) | (min) | (ng · min/mL) | | |
| Example 3 | 1 | 0 | 1806 | 1926 | 1755 | 1374 | 894 | 774 | 707 | 333 | 1926 | 10.0 | 32250 | 47895 | 191492 |
| | 2 | 0 | 1195 | 1844 | 1476 | 1029 | 800 | 605 | 503 | 222 | 1844 | 10.0 | 27185 | 39710 | 148349 |
| | Mean | 0 | 1501 | 1885 | 1616 | 1202 | 847 | 689 | 605 | 277 | 1885 | 10.0 | 29718 | 43803 | 169920 |
| Example 10 | 1 | 0 | 440 | 864 | 988 | 709 | 417 | 308 | 246 | 169 | 988 | 20.0 | 13620 | 22105 | 83080 |
| | 2 | 0 | 454 | 974 | 1170 | 964 | 568 | 441 | 404 | 229 | 1170 | 20.0 | 15425 | 26095 | 114865 |
| | Mean | 0 | 447 | 919 | 1079 | 837 | 493 | 375 | 325 | 199 | 1079 | 20.0 | 14523 | 24100 | 98973 |
| Example 11 | 1 | 0 | 1120 | 1500 | 1580 | 1360 | 815 | 636 | 570 | 301 | 1580 | 20.0 | 24750 | 39450 | 164190 |
| | 2 | 0 | 1630 | 2350 | 2280 | 1930 | 1250 | 878 | 665 | 357 | 2350 | 10.0 | 37175 | 58225 | 222310 |
| | Mean | 0 | 1375 | 1925 | 1930 | 1645 | 1033 | 757 | 618 | 329 | 1965 | 15.0 | 30963 | 48838 | 193250 |
| Comp. Example 12 | 1 | 0 | 0 | 0 | 11 | 12 | 54 | 302 | 308 | 150 | 308 | 120.0 | 54 | 165 | 43116 |
| | 2 | 0 | 15 | 24 | 26 | 50 | 343 | 288 | 291 | 165 | 343 | 60.0 | 387 | 768 | 52173 |
| | Mean | 0 | 8 | 12 | 18 | 31 | 199 | 295 | 299 | 158 | 325 | 90.0 | 220 | 466 | 47645 |
| Comp. Example 13 | 1 | 0 | 547 | 725 | 471 | 358 | 204 | 147 | 118 | 80 | 725 | 10.0 | 10527 | 14669 | 44204 |
| | 2 | 0 | 772 | 1087 | 864 | 742 | 516 | 384 | 293 | 174 | 1087 | 10.0 | 16334 | 24363 | 94925 |
| | Mean | 0 | 660 | 906 | 667 | 550 | 360 | 266 | 206 | 127 | 906 | 10.0 | 13430 | 19516 | 69564 |
| Comp. Example 14 | 1 | 0 | 668 | 882 | 607 | 430 | 305 | 232 | 215 | 140 | 882 | 10.0 | 12987 | 18175 | 65235 |
| | 2 | 0 | 601 | 1093 | 1045 | 872 | 598 | 514 | 410 | 185 | 1093 | 10.0 | 16430 | 26016 | 114278 |
| | Mean | 0 | 634 | 987 | 826 | 651 | 452 | 373 | 312 | 162 | 987 | 10.0 | 14708 | 22096 | 89756 |
| Comp. Example 15 | 1 | 0 | 203 | 561 | 710 | 633 | 389 | 283 | 266 | 139 | 710 | 20.0 | 8770 | 15482 | 72684 |
| | 2 | 0 | 567 | 855 | 1000 | 978 | 901 | 748 | 702 | 342 | 1000 | 20.0 | 14245 | 24133 | 161381 |
| | Mean | 0 | 385 | 708 | 855 | 805 | 645 | 515 | 479 | 240 | 855 | 20.0 | 11507 | 19807 | 117033 |
| Comp. Example 16 | 1 | 0 | 401 | 799 | 707 | 606 | 390 | 335 | 317 | 141 | 799 | 10.0 | 11531 | 18092 | 81137 |
| | 2 | 0 | 485 | 979 | 1142 | 1069 | 620 | 489 | 398 | 187 | 1142 | 20.0 | 15482 | 26537 | 116933 |
| | Mean | 0 | 443 | 889 | 924 | 837 | 505 | 412 | 357 | 164 | 971 | 15.0 | 13506 | 22314 | 99035 |
| Comp. Example 17 | 1 | 0 | 169 | 378 | 567 | 451 | 242 | 289 | 290 | 300 | 567 | 20.0 | 6515 | 11606 | 74055 |
| | 2 | 0 | 134 | 256 | 377 | 369 | 260 | 211 | 224 | 111 | 377 | 20.0 | 4476 | 8204 | 51356 |
| | Mean | 0 | 152 | 317 | 472 | 410 | 251 | 250 | 257 | 205 | 472 | 20.0 | 5495 | 9905 | 62705 |
| Comp. Example 18 | 1 | 0 | 20 | 30 | 42 | 65 | 483 | 581 | 407 | 157 | 581 | 90.0 | 533 | 1068 | 73909 |
| | 2 | 0 | 0 | 25 | 41 | 59 | 86 | 144 | 158 | 142 | 158 | 120.0 | 391 | 888 | 29015 |
| | Mean | 0 | 10 | 27 | 41 | 62 | 285 | 363 | 283 | 149 | 370 | 105.0 | 462 | 978 | 51462 |
| Comp. Example 19 | 1 | 0 | 445 | 846 | 756 | 684 | 413 | 331 | 344 | 156 | 846 | 10.0 | 12352 | 19552 | 87279 |
| | 2 | 0 | 904 | 1524 | 1259 | 925 | 660 | 506 | 383 | 174 | 1524 | 10.0 | 22245 | 33166 | 121158 |
| | Mean | 0 | 675 | 1185 | 1007 | 805 | 537 | 419 | 363 | 165 | 1185 | 10.0 | 17299 | 26359 | 104218 |
| Comp. Example 20 | 1 | 0 | 684 | 1073 | 1204 | 1257 | 803 | 534 | 416 | 193 | 1257 | 30.0 | 17488 | 29793 | 131526 |
| | 2 | 0 | 1058 | 1921 | 1391 | 1045 | 640 | 540 | 428 | 199 | 1921 | 10.0 | 26653 | 38833 | 133958 |
| | Mean | 0 | 871 | 1497 | 1298 | 1151 | 721 | 537 | 422 | 196 | 1589 | 20.0 | 22070 | 34313 | 132742 |

For measurement of the plasma baclofen concentration, blood was collected from the femoral vein and collected into test tube with EDTA-2K. The plasma baclofen concentrations were measured by LC-MS/MS method.

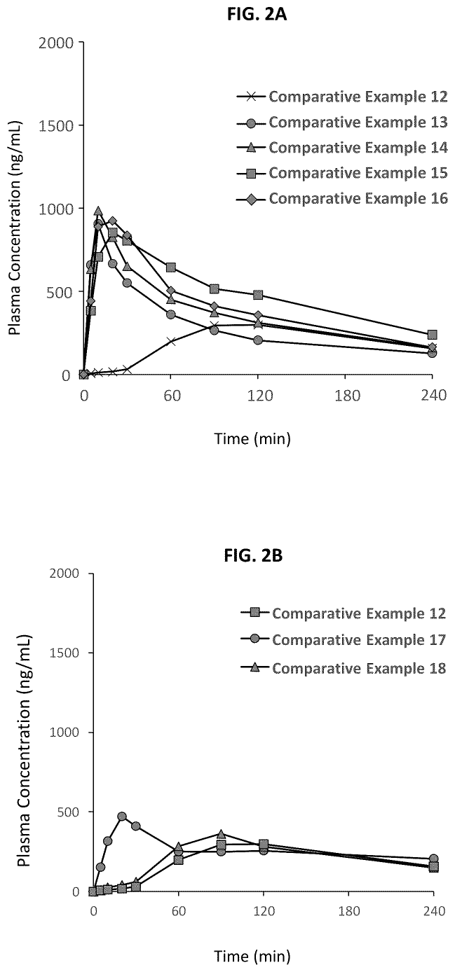

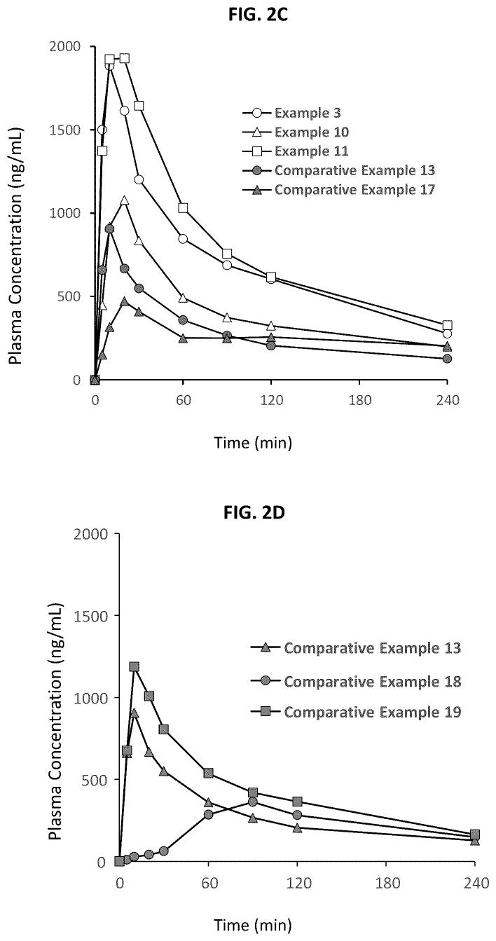

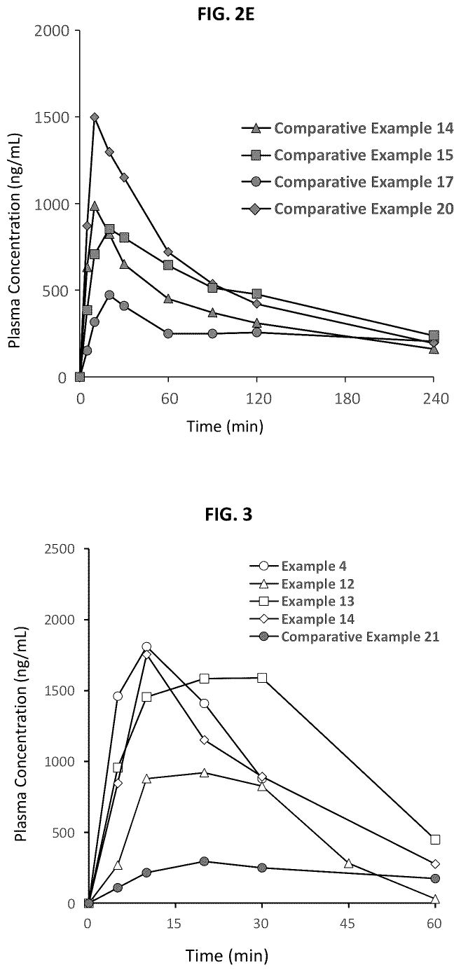

The plasma baclofen concentration—time profiles, and the corresponding plasma baclofen concentrations and PK parameters are shown in FIGS. 2A-2E and TABLE 4, respectively. As shown in FIG. 2A, baclofen compositions comprising absorption enhancers in Comparative Examples 13-16 had higher baclofen concentrations than the composition without an enhancer in Comparative Example 12. The absorption enhancers in Comparative Examples 13-16 had comparable effects on absorption enhancement. As shown in FIG. 2B, a slight effect of MCC on absorption enhancement was observed in Comparative Example 17. There was no effect of lactose on absorption enhancement observed in Comparative Example 18. As shown in FIG. 2C, the baclofen compositions in Examples 3, 10 and 11 having a combination of alkanoylcholines (e.g., LCC, PCC or MyCC) and MCC drastically increased plasma baclofen concentration. As shown in FIG. 2D, the baclofen formulation in Comparative Example 19, comprising LCC and lactose, increased plasma baclofen concentration to a lesser degree than the composition comprising LCC and MCC. As shown in FIG. 2E, the compositions comprising both SLS and MCC (Comparative Example 20) also enhanced baclofen absorption to a lesser degree than the formulation comprising LCC and MCC. The baclofen composition comprising both LCC and MCC showed the most improved absorption in this example.

As shown in TABLE 4, the AUCs for the baclofen composition comprising both an alkanoylcholine (e.g., LCC) and MCC were much higher than those for other compositions without the alkanoylcholine and MCC combination. The AUC_{0-20 min} and AUC_{0-30 min} for the composition in Example 3 were about 5.4 and 4.4 times, respectively, those of the composition in Comparative Example 17. Other PK parameters, such as Cmax and AUC observed at later time points (AUC_{0-60 min}, AUC_{0-90 min}, AUC_{0-240 min}), are much higher for the baclofen compositions comprising both an alkanoylcholine (e.g., LCC) and MCC compared to other compositions.

### Example 4: Assessing Absorption of Levodopa Intranasal Powder Compositions in Monkeys

TABLE 5
Plasma levodopa concentrations and pharmacokinetic parameters after nasally administrating levodopa preparations

| | | Time (min)/Plasma Concentration (ng/mL) | | | | | | | | | Cmax | Tmax | AUC₀₋₂₀ | AUC₀₋₃₀ | AUC₀₋₆₀ |
|---|---|---|---|---|---|---|---|---|---|---|---|---|---|---|---|
| | | 0 | 5 | 10 | 20 | 30 | 45 | 60 | — | — | ng/mL | min | ng · min/mL | | |
| Example 4 | 1 | 0 | 1540 | 2100 | 1510 | 1040 | — | 301 | — | — | 2100 | 10.0 | 31000 | 43750 | 63865 |
| | 2 | 0 | 1380 | 1520 | 1310 | 718 | — | 209 | — | — | 1520 | 10.0 | 24850 | 34990 | 48895 |
| | Mean | 0 | 1460 | 1810 | 1410 | 879 | — | 255 | — | — | 1810 | 10.0 | 27925 | 39370 | 56380 |
| Example 12 | 1 | 0 | 279 | 832 | 723 | 643 | — | 174 | | | 832 | 10.0 | 11250 | 18080 | 30335 |
| | 2 | 0 | 259 | 927 | 1120 | 1010 | — | 390 | | | 1120 | 20.0 | 13848 | 24498 | 45498 |
| | Mean | 0 | 269 | 880 | 922 | 827 | — | 282 | | | 976 | 15.0 | 12549 | 21289 | 37916 |
| Example 13 | 1 | 0 | 603 | 1010 | 1300 | 1600 | — | 414 | | | 1600 | 30.0 | 17090 | 31590 | 61800 |
| | 2 | 0 | 1310 | 1900 | 1870 | 1580 | — | 484 | | | 1900 | 10.0 | 30150 | 47400 | 78360 |
| | Mean | 0 | 957 | 1455 | 1585 | 1590 | — | 449 | | | 1750 | 20.0 | 23620 | 39495 | 70080 |
| Example 14 | 1 | 0 | 1110 | 2310 | 1780 | 1510 | — | 510 | | | 2310 | 10.0 | 31775 | 48225 | 78525 |
| | 2 | 0 | 584 | 1200 | 526 | 278 | — | 44 | | | 1200 | 10.0 | 14550 | 18570 | 23406 |
| | Mean | 0 | 847 | 1755 | 1153 | 894 | — | 277 | | | 1755 | 10.0 | 23163 | 33398 | 50966 |
| Comp. Example 21 | 1 | 0 | 110 | 180 | 260 | 230 | 170 | 150 | — | — | 260 | 20.0 | 3200 | 5650 | 11050 |
| | 2 | 0 | 110 | 250 | 330 | 270 | 180 | 140 | — | — | 330 | 20.0 | 4075 | 7075 | 12850 |
| | Mean | 0 | 110 | 215 | 295 | 250 | 175 | 145 | — | — | 295 | 20.0 | 3638 | 6363 | 11950 |

TABLE 5 shows examples with levodopa. 50 mg of Examples 4, 12 to 14 were delivered into the right nasal cavity [50 mg (an equivalent dose of 20 mg levodopa)/nostril/body] and 100 mg of Comparative Example 21 was delivered into both nasal cavities [100 mg (an equivalent dose of 50 mg levodopa)/both nostril/body=50 mg (an equivalent dose of 25 mg levodopa)/nostril×both nostril], of conscious male cynomolgus monkeys, with the intranasal delivery device for powder formulation which is composed of a nozzle, a container for a unit dose of powder formulation and an air generating unit. For measurement of the plasma levodopa concentration, blood was collected from the femoral vein and collected into test tube with EDTA-2K. 25% Sodium pyrosulfite solution and the plasma is mixed in a volumetric ratio of 1:50. The plasma levodopa concentrations were measured by LC-MS/MS method.

The plasma levodopa concentration—time profiles, and the corresponding plasma levodopa concentrations and PK parameters are shown in FIG. 3 and TABLE 5, respectively. As shown in FIG. 3, the levodopa composition in Examples 4, 12 to 14, which comprise a combination of a choline ester (e.g., LCC, PCC, or MyCC) and MCC, drastically increased the plasma levodopa concentration when compared with the composition in Comparative Example 21, which only had MCC. This significant increase of plasma concentration was observed despite of a levodopa dose of 20 mg (40% of 50 mg of powder composition administered) in Example 4 compared to a 50 mg levodopa dose (50% of 100 mg of powder composition administered) in Comparative Example 21.

AUC_{0-20 min} and AUC_{0-30 min} values indicate absorbability in an early period after dosing. As shown in Table 5, the AUC_{0-20 min} and AUC_{0-30 min} for the levodopa composition comprising MCC and LCC were drastically higher—7.7 and 6.1 times, respectively—than those of the levodopa composition without LCC. The AUC_{0-20 min} and AUC_{0-30 min} for the levodopa composition comprising the choline ester PCC and MCC were about 3.4 times higher than those of the levodopa composition without PCC. Other PK parameters, such as Cmax and AUC observed at later time points (AUC_{0-60 min}, AUC_{0-90 min}, AUC_{0-240 min} and AUC_{0-480 min}), are much higher for the levodopa compositions comprising both an alkanoylcholine and MCC compared to those with MCC alone.

Concentration of levodopa may also be measured in the cerebrospinal fluid (CSF) of a subject. CSF samples may be obtained from the cerebellomedullary cistern of an animal (e.g., cynomolgus monkey) under anesthesia with isoflurane. CSF samples may be obtained from a human subject via a spinal tap. Similarly, CSF levodopa concentrations may be measured by LC-MS/MS method.

### Example 5: Assessing Absorption of Calcitonin Intranasal Powder Compositions in Monkeys

TABLE 6 shows examples with calcitonin. 60 mg of each Example 5 and Comparative Example 22 were delivered into the right nasal cavity of conscious male cynomolgus monkeys, with an intranasal delivery device for powder formulation (at an equivalent dose of 0.1 mg calcitonin). For measurement of the plasma calcitonin concentration, blood was collected from the femoral vein and collected into test tube with EDTA-2K. The plasma calcitonin concentrations were measured by ELISA.

TABLE 6
Plasma calcitonin concentrations and pharmacokinetic parameters after nasally administrating calcitonin preparations

| | Time (min)/Plasma Concentration (ng/mL) | | | | | | | | | | | Cmax | Tmax | AUC₀₋₂₀ | AUC₀₋₃₀ | AUC₀₋₁₂₀ |
|---|---|---|---|---|---|---|---|---|---|---|---|---|---|---|---|---|
| | | 0 | 5 | 10 | 20 | 30 | 60 | 90 | 120 | — | — | (ng/mL) | (min) | (ng · min/mL) | | |
| Example 5 | 1 | 0.0 | 9.6 | 13.3 | 13.7 | 10.2 | 5.9 | 2.9 | 1.9 | — | — | 13.7 | 20.0 | 216.8 | 336.2 | 779.0 |
| | 2 | 0.0 | 31.0 | 35.4 | 21.2 | 14.2 | 4.2 | 1.9 | 1.0 | — | — | 35.4 | 10.0 | 526.6 | 704.0 | 1116.9 |
| | Mean | 0.0 | 20.3 | 24.4 | 17.5 | 12.2 | 5.0 | 2.4 | 1.5 | — | — | 24.5 | 15.0 | 371.7 | 520.1 | 947.9 |
| Comp. Example 22 | 1 | 0.0 | 5.9 | 9.0 | 8.3 | 5.2 | 1.2 | 0.5 | 0.2 | — | — | 9.0 | 10.0 | 138.7 | 206.4 | 338.7 |
| | 2 | 0.0 | 5.5 | 10.2 | 9.1 | 7.7 | 1.9 | 0.7 | 0.4 | — | — | 10.2 | 10.0 | 149.1 | 232.8 | 430.8 |
| | Mean | 0.0 | 5.7 | 9.6 | 8.7 | 6.4 | 1.5 | 0.6 | 0.3 | — | | 9.6 | 10.0 | 143.9 | 219.6 | 384.7 |

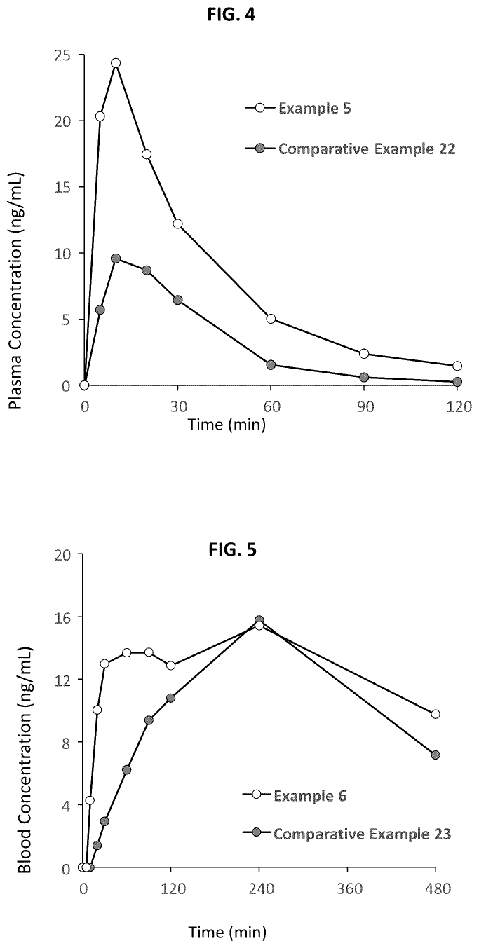

The plasma calcitonin concentration—time profiles, and the corresponding plasma calcitonin concentrations and PK parameters are shown in FIG. 4 and TABLE 6, respectively.

As shown in FIG. 4, the calcitonin composition in Example 5 comprises a combination of LCC and MCC and it drastically increased the plasma calcitonin concentration when compared with the composition in Comparative Example 22, which only contained MCC.

As is shown in TABLE 6, AUC_{0-20 min} and AUC_{0-30 min} for Example 5 consisting of calcitonin, LCC and MCC, was drastically higher (about 2.6 and 2.3 times, respectively) than that for Comparative Example 22 consisting of calcitonin and MCC alone. Other PK parameters, such as Cmax and AUC observed at later time points (AUC_{0-60 min}, AUC_{0-90 min} AUC_{0-240 min}, AUC_{0-480 min}), are much higher for the calcitonin compositions comprising both LCC and MCC compared to other compositions.

### Example 6: Assessing Absorption of Cyclosporine A Intranasal Powder Compositions in Monkeys

TABLE 7 shows example with cyclosporine A. 60 mg of each Example 6 and Comparative Example 23 were delivered into the right nostrils of conscious male cynomolgus monkeys with an intranasal delivery device (at an equivalent dose of 30 mg cyclosporine A). For measurement of the plasma cyclosporin A concentration, blood was collected from the femoral and collected into test tube with EDTA-2K. The blood cyclosporin A concentrations were measured by LC-MS/MS method.

TABLE 7
Plasma cyclosporine A concentrations and pharmacokinetic parameters after nasally administrating cyclosporine A preparations

| | | Time (min)/Plasma Concentration (ng/mL) | | | | | | | | | | Cmax | Tmax | AUC₀₋₂₀ | AUC₀₋₃₀ | AUC₀₋₄₈₀ |
|---|---|---|---|---|---|---|---|---|---|---|---|---|---|---|---|---|
| | | 0 | 5 | 10 | 20 | 30 | 60 | 90 | 120 | 240 | 480 | ng/mL | min | ng · min/mL | | |
| Example 6 | 1 | 0.0 | 0.0 | 4.5 | 11.2 | 14.6 | 14.3 | 13.3 | 12.8 | 13.5 | 11.2 | 14.6 | 30.0 | 89.3 | 218.1 | 5989.2 |
| | 2 | 0.0 | 0.0 | 4.1 | 8.9 | 11.4 | 13.1 | 14.1 | 12.9 | 17.4 | 8.3 | 17.4 | 240.0 | 75.2 | 176.5 | 6259.1 |
| | Mean | 0.0 | 0.0 | 4.3 | 10.0 | 13.0 | 13.7 | 13.7 | 12.9 | 15.4 | 9.8 | 16.0 | 135.0 | 82.2 | 197.3 | 6124.2 |
| Comp. Example 23 | 1 | 0.0 | 0.0 | 0.0 | 1.4 | 3.2 | 5.2 | 6.8 | 6.3 | 18.4 | 5.9 | 18.4 | 240.0 | 7.0 | 30.0 | 4934.3 |
| | 2 | 0.0 | 0.0 | 0.0 | 1.4 | 2.7 | 7.2 | 12.0 | 15.3 | 13.1 | 8.4 | 15.3 | 120.0 | 7.2 | 27.7 | 5157.6 |
| | Mean | 0.0 | 0.0 | 0.0 | 1.4 | 2.9 | 6.2 | 9.4 | 10.8 | 15.8 | 7.2 | 16.9 | 180.0 | 7.1 | 28.8 | 5045.9 |

The blood cyclosporin A concentration—time profiles, and the corresponding blood cyclosporine A concentrations and PK parameters are shown in FIG. 5 and TABLE 7, respectively. As shown in FIG. 5, the cyclosporin A composition in Example 6 comprises a combination of LCC and MCC and it drastically increased plasma cyclosporin A concentration until 240 minutes after dosing when compared with the Comparative Example 23.

As shown in TABLE 7, AUC_{0-20 min} and AUC_{0-30 min} value for Example 6 consisting of cyclosporine A, LCC and MCC, was drastically higher (about 11.6 times and 6.9 times, respectively) than that for Comparative Example 23 consisting of cyclosporine A and MCC (without alkanoylcholines). Other PK parameters, such as Cmax and AUC observed at later time points (AUC₀₋₆₀ min, AUC_{0-90 min} AUC_{0-240 min}, AUC_{0-480 min}), are much higher for the cyclosporine A compositions comprising both LCC and MCC compared to other compositions.

### Example 7: Assessing Absorption of Antisense Oligomer Intranasal Powder Compositions in Monkeys

TABLE 8 shows examples with antisense oligonucleotide. 70 mg of each Example 7 and Comparative Example 24 with antisense oligonucleotide were delivered into the right nasal cavity of conscious male cynomolgus monkeys with an intranasal delivery device for powder formulation (at an equivalent dose of 23 mg antisense oligonucleotide). For measurement of the plasma antisense oligonucleotide concentration, blood was collected at different time points from the femoral vein and collected into test tube with EDTA-2K. The plasma antisense oligonucleotide concentrations were measured by LC-MS/MS.

TABLE 8
Plasma antisense oligonucleotide concentrations and pharmacokinetic parameters
after nasally administrating antisense oligonucleotide preparations.

| | | Time (min)/Plasma Concentration (ng/mL) | | | | | | | | | | Cmax | Tmax | AUC₀₋₂₀ | AUC₀₋₃₀ | AUC₀₋₄₈₀ |
|---|---|---|---|---|---|---|---|---|---|---|---|---|---|---|---|---|
| | | 0 | 5 | 10 | 20 | 30 | 60 | 90 | 120 | 240 | 480 | ng/mL | min | ng · min/mL | | |
| Example 7 | 1 | 0 | 233 | 390 | 508 | 348 | 188 | 101 | 52 | 9 | 4 | 508 | 20.0 | 6630 | 10910 | 30815 |
| | 2 | 0 | 406 | 700 | 1010 | 1060 | 818 | 677 | 491 | 199 | 28 | 1060 | 30.0 | 12330 | 22680 | 159399 |
| | Mean | 0 | 320 | 545 | 759 | 704 | 503 | 389 | 271 | 104 | 16 | 784 | 25.0 | 9480 | 16795 | 95107 |
| Comparative | 1 | 0 | 86 | 219 | 473 | 514 | 394 | 282 | 188 | 55 | 27 | 514 | 30.0 | 4439 | 9374 | 64580 |
| Example 24 | 2 | 0 | 88 | 189 | 319 | 330 | 208 | 139 | 95 | 31 | 20 | 330 | 30.0 | 3453 | 6698 | 37184 |
| | Mean | 0 | 87 | 204 | 396 | 422 | 301 | 211 | 141 | 43 | 24 | 422 | 30.0 | 3946 | 8036 | 50882 |

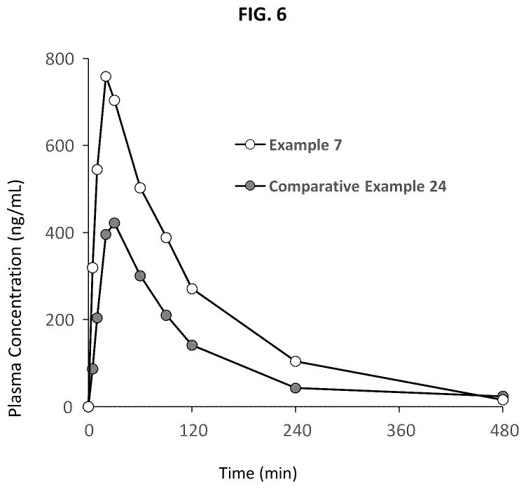

The plasma antisense oligonucleotide concentration—time profiles, and the corresponding plasma calcitonin concentrations and PK parameters are shown in FIG. 6 and TABLE 8, respectively. As shown in FIG. 6, the composition in Example 7 which comprises a combination of LCC and MCCs and it drastically increased plasma antisense oligonucleotide concentration after dosing when compared with the Comparative Example 24, which includes MCC and TCP.

As is shown in TABLE 8, AUC_{0-30 min} value for Example 7 consisting of antisense oligonucleotide, LCC, MCC and TCP, was drastically higher (2.1 times) than that for Comparative Example 24 consisting of antisense oligonucleotide, MCC and TCP (without alkanoylcholines). AUC_{0-20 min} for Example 7 was also drastically higher (2.4 times) than that for Comparative Example 24.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1.-104. (canceled)

105. A method of treating a disease or condition in a subject in need thereof, said method comprising administering to said subject a pharmaceutical composition comprising an active agent for treating Parkinson's disease, and an absorption enhancer comprising a choline ester.

106.-117. (canceled)

118. A pharmaceutical composition comprising:
a) an active ingredient;
b) an absorption enhancer comprising a choline ester; and
c) an excipient comprising a microcrystalline cellulose;
wherein the pharmaceutical composition is formulated in a solid dosage form.

119. The pharmaceutical composition of claim 118, wherein the active ingredient comprises a peptide, a small molecule drug, a middle molecule drug, or a nucleic acid molecule.

120. The pharmaceutical composition of claim 118, wherein the active ingredient comprises epinephrine or a pharmaceutically acceptable salt thereof, baclofen or a pharmaceutically acceptable salt thereof, levodopa or a pharmaceutically acceptable salt thereof, calcitonin or a pharmaceutically acceptable salt thereof, cyclosporine A or a pharmaceutically acceptable salt thereof, an antisense oligonucleotide or a pharmaceutically acceptable salt thereof, or any combination thereof.

121. The pharmaceutical composition of claim 120, wherein the active ingredient comprises epinephrine or a pharmaceutically acceptable salt thereof.

122. The pharmaceutical composition of claim 120, wherein the active ingredient comprises baclofen or a pharmaceutically acceptable salt thereof.

123. The pharmaceutical composition of claim 120, wherein the active ingredient comprises levodopa or a pharmaceutically acceptable salt thereof.

124. The pharmaceutical composition of claim 120, wherein the active ingredient comprises calcitonin or a pharmaceutically acceptable salt thereof.

125. The pharmaceutical composition of claim 120, wherein the active ingredient comprises cyclosporine A or a pharmaceutically acceptable salt thereof.

126. The pharmaceutical composition of claim 120, wherein the active ingredient comprises an antisense oligonucleotide or a pharmaceutically acceptable salt thereof.

127. The pharmaceutical composition of claim 118, wherein the choline ester comprises a C₈ to C₂₀ alkanoylcholine selected from the group consisting of: an arachidoyl choline or a pharmaceutically acceptable salt thereof, a stearoyl choline or a pharmaceutically acceptable salt thereof, a palmitoyl choline or a pharmaceutically acceptable salt thereof, myristoylcholine or a pharmaceutically acceptable salt thereof, a lauroylcholine or a pharmaceutically acceptable salt thereof, a caproylcholine or a pharmaceutically acceptable salt thereof, a capryloyl choline or a pharmaceutically acceptable salt thereof, and any combination thereof.

128. The pharmaceutical composition of claim 118, wherein the choline ester comprises a lauroylcholine or a pharmaceutically acceptable salt thereof.

129. The pharmaceutical composition of claim 118, further comprising an additional excipient.

130. The pharmaceutical composition of claim 128, wherein the additional excipient comprises a sugar alcohol.

131. The pharmaceutical composition of claim 118, wherein when the pharmaceutical composition is administered to a subject, a pharmacokinetic parameter of the active ingredient or a metabolite thereof, improves by at least about 15% compared to a corresponding composition that does not comprise the absorption enhancer.

132. The pharmaceutical composition of claim 131, wherein the improved pharmacokinetic parameter of the active ingredient or a metabolite thereof, comprises a shorter time to reach a maximum drug concentration (Tₘₐₓ).

133. The pharmaceutical composition of claim 131, wherein the improved pharmacokinetic parameter of the active ingredient or a metabolite thereof, comprises a larger area under curve (AUC) from 0-20 minutes (AUC_{0-20 min}).

134. The pharmaceutical composition of claim 118, wherein the absorption enhancer is present in the pharmaceutical composition in an amount of about 1 mg to about 5 mg.

135. The pharmaceutical composition of claim 118, wherein the microcrystalline cellulose is present in an amount of from about 40% to about 90% by weight, based on a total weight of the pharmaceutical composition.

136. The pharmaceutical composition of claim 118, wherein the solid dosage form comprises a powder dosage form.
